(19) United States
(12) Patent Application Publication
(10) Pub. No.: US 2025/0308709 A1
(43) Pub. Date: Oct. 2, 2025

(54) SYSTEM AND METHOD FOR PREDICTING INSULIN RESISTANCE OR PANCREATIC BETA-CELL FUNCTION AND COMPUTER READABLE MEDIUM THEREOF

(71) Applicant: Taichung Veterans General Hospital, Taichung City (TW)

(72) Inventors: Chia-Lin Li, Tainan City (TW); Wei-Ju Liu, Tainan City (TW)

(21) Appl. No.: 18/616,217

(22) Filed: Mar. 26, 2024

(51) Int. Cl. G16H 50/70; A61B 5/145; G06N 5/022; G06N 20/00; G16H 50/50

(52) U.S. Cl. CPC G16H 50/70; G06N 5/022; G06N 20/00; G16H 50/50; A61B 5/14532; A61B 5/14546

(57) ABSTRACT

A system and a method for predicting insulin resistance and/or pancreatic β-cell function are provided, where a machine learning model is utilized to predict insulin resistance and/or pancreatic a decline of β-cell function of a subject in need thereof based on a feature set extracted from a database. Therefore, clinicians or the subject can be warned to take necessary actions on, and adjust related medical treatment or lifestyle before the subject is diagnosed with diabetes mellitus. In addition, a computer readable medium thereof is also provided.

### BACKGROUND OF THE INVENTION

### 1. Field of the Invention

The present disclosure relates to medical monitoring application, and more particularly to prediction of insulin resistance and/or pancreatic β-cell function.

### 2. Description of the Prior Art

Insulin resistance (IR) is a condition where effect of the insulin is reduced in cells of the skeletal muscle, liver, and adipose tissue. IR arises when a given concentration of insulin is associated with an insufficient glucose response. There are a number of underlying factors that may contribute to the development of IR, including obesity, stress, medication (e.g., steroid), pregnancy, insulin antibodies, and genetic defects in insulin signaling pathways. IR is also associated with clinical diseases, such as diabetes mellitus (DM), coronary artery disease, metabolic syndrome, polycystic ovary syndrome, and nonalcoholic fatty liver disease.

IR is well-known as an indicator for early diagnosis of DM.

One of the first of manifestations of metabolic syndrome is the occurrence of IR. The decreased effect of insulin (i.e. IR) leads to overwork of β cells which secrete much more insulin as a compensatory mechanism to maintain plasma glucose level. As the metabolic syndrome gets worse, the numbers of β cells decrease and the level of the plasma glucose start to increase. Finally, the increased plasma glucose meets the diagnostic definition of DM. It is noteworthy that a decline in β-cell function (decreased secretion of insulin) begins as early as 12 years before DM diagnosis and continues throughout the disease process. Many clinical studies have suggested that the earlier intervention for DM, the better outcome of DM; and early control of DM has great benefits in reducing the dysglycemic legacy effect (i.e. metabolic memory). Hence, identifying IR (i.e. the earliest finding of DM) or β-cell function is important in the clinical spectrum of DM.

In clinical practice, static testing is the Homeostasis Model Assessment of IR (HOMA-IR) and β-cell function (HOMA-β). HOMA estimates the degree of β-cell deficiency and insulin sensitivity based on an equation that consists of the concentration of fasting plasma insulin and fasting plasma glucose. However, fasting plasma insulin level is not routinely checked; but the fasting plasma insulin level is needed for HOMA-IR and HOMA-β calculation. As a consequence, IR and β-cell deficiency cannot be well utilized as indicators in clinical practice despite its important clinical implications.

Recently, machine learning has been used to deal with this situation. However, most of the studies are based on prediction models in the field of DM instead of the indicators (i.e., IR and decline in β-cell function) for early diagnosis of DM in non-diabetic patients.

In view of the foregoing, there is an unmet need in the art to utilize artificial intelligence for predicting IR and/or β-cell function in non-diabetic population.

### SUMMARY OF THE INVENTION

To solve the aforementioned problems, the present disclosure provides a system for predicting insulin resistance and/or pancreatic β-cell function of a subject in need thereof, comprising: a database configured to provide a data set; a feature extraction module configured to collect and process features from the data set to generate a feature set regarding the subject, wherein the feature set comprises age, gender, race, and body mass index of the subject; and a model building and optimization module configured to build a machine learning model based on the feature set to predict the insulin resistance and/or the pancreatic β-cell function of the subject.

The present disclosure further provides a method for predicting insulin resistance and/or pancreatic β-cell function of a subject in need thereof, comprising: configuring a database to provide a data set; configuring a feature extraction module to collect and process features from the data set to generate a feature set regarding the subject, wherein the feature set comprises age, gender, race, and body mass index of the subject; and configuring a model building and optimization module to build a machine learning model based on the feature set to predict the insulin resistance and/or the pancreatic β-cell function of the subject.

The present disclosure further provides a computer readable medium storing a computer executable code, upon executed, the computer executable code implement the method of the present disclosure.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating an exemplifying structure of the system and method for predicting insulin resistance and/or pancreatic β-cell function in accordance with embodiments of the present disclosure.

FIG. 2A is a curve graph illustrating receiver operating characteristic (ROC) curve in the training group for predicting insulin resistance (IR) in patients with chronic kidney disease (CKD) by XGboost, random forests, logistic regression, and deep neural network (DNN) in accordance with embodiments of the present disclosure.

FIG. 2B is a curve graph illustrating receiver operating characteristic (ROC) curve in the internal validation group for predicting insulin resistance (IR) in patients with chronic kidney disease (CKD) by XGboost, random forests, logistic regression, and deep neural network (DNN) in accordance with embodiments of the present disclosure.

FIG. 2C is a curve graph illustrating receiver operating characteristic (ROC) curve in the test group for predicting insulin resistance (IR) in patients with chronic kidney disease (CKD) by XGboost, random forests, logistic regression, and deep neural network (DNN) in accordance with embodiments of the present disclosure.

FIG. 3A is a bar graph illustrating the detailed features importance of the XGboost prediction model in accordance with embodiments of the present disclosure.

FIG. 3B is a bar graph illustrating the detailed features importance of the random forests prediction model in accordance with embodiments of the present disclosure.

FIG. 4A is a heat graph illustrating positive and negative impact explanation of features for predicting insulin resistance using SHAP (SHapley Additive exPlanations) values of the XGboost prediction model in accordance with embodiments of the present disclosure.

FIG. 4B is a heat graph illustrating positive and negative impact explanation of features for predicting insulin resistance using SHAP values of the random forests prediction model in accordance with embodiments of the present disclosure.

FIG. 5A and FIG. 5B are Kaplan-Meier survival curve of CV mortality according to insulin resistance or not, illustrating clinical implication (cardiovascular disease (CV) mortality and all-cause mortality) predicted by XGboost prediction model from the Taiwan biobank database in accordance with embodiments of the present disclosure.

FIG. 5C and FIG. 5D are Kaplan-Meier survival curve of all-cause mortality according to insulin resistance or not, illustrating clinical implication (cardiovascular disease (CV) mortality and all-cause mortality) predicted by XGboost prediction model from the Taiwan biobank database in accordance with embodiments of the present disclosure.

FIG. 6A is a bar graph illustrating personalized IR risk and explanation of a feature set in accordance with embodiments of the present disclosure.

FIG. 6B is a bar graph illustrating personalized β-cell deficiency risk and explanation of a feature set in accordance with embodiments of the present disclosure.

### DETAILED DESCRIPTION

The following embodiments are provided to illustrate the present disclosure in detail. A person having ordinary skill in the art can easily understand the advantages and effects of the present disclosure after reading this disclosure, and also can implement or apply in other different embodiments. Therefore, any element or method within the scope of the present disclosure disclosed herein can combine with any other element or method disclosed in any embodiment of the present disclosure.

The relationships, structures, steps, factors, and other features shown in accompanying drawings of this disclosure are only used to illustrate embodiments described herein, such that those with ordinary skill in the art can read and understand the present disclosure therefrom, of which are not intended to limit the scope of this disclosure. Any changes, modifications, or adjustments of said features, without affecting the designed purposes and effects of the present disclosure, should all fall within the scope of technical content of this disclosure.

As used herein, when describing an object “comprises,” “includes” or “has” a limitation, unless otherwise specified, it may additionally encompass other modules, elements, components, structures, parts, devices, systems, steps, connections, factors, features etc., and should not exclude others.

As used herein, sequential terms, such as “first,” “second,” “third,” “fourth,” etc., are only cited in convenience of describing or distinguishing limitations such as modules, sets, databases, elements, components, structures, parts, devices, systems, steps, connections, factors, or features from one another, which are not intended to limit the scope of this disclosure, nor to limit spatial sequences between such limitations. Further, unless otherwise specified, wordings in singular forms such as “a,” “an” and “the” also pertain to plural forms, and wordings such as “or” and “and/or” may be used interchangeably.

As used herein, the terms “subject,” “participant,” “individual,” and “patient” may be interchangeable.

As used herein, the terms “fasting blood glucose” and “fasting plasma glucose” may be interchangeable.

As used herein, the terms “decline in β-cell function,” “decline of β-cell function,” and “β-cell deficiency” may be interchangeable.

As used herein, the terms “variable,” “factor,” and “feature” may be interchangeable.

As used herein, the terms “comprise,” “comprising,” “include,” “including,” “have,” “having,” “contain,” “containing,” or any other variations thereof are intended to cover a non-exclusive inclusion. For example, a module, a process or a method that comprises a list of elements is not necessarily limited to only those elements, but may include other elements not expressly listed, or inherent to such module, process, or method.

The terms “Homeostasis Model Assessment of IR (HOMA-IR)” and “Homeostasis Model Assessment of β-cell function (HOMA-R)” used herein are used for assessing IR and β-cell function. HOMA-IR and HOMA-β are the most acceptable method for estimating IR and a decline of β-cell function. Specifically, variables for HOMA-IR calculation include age, sex, race, year of assessment, BMI, and smoking status. IR (i.e. increased HOMA-IR). In addition, β-cell function has a strong association with development of type 2 diabetes mellitus (DM). The association is statistically independent of impaired glucose tolerance status, obesity, and body fat distribution. High values of HOMA-IR and HOMA-β are independently associated with high risks of developing prediabetes. HOMA-IR adopts the following formula to index insulin resistance: fasting plasma insulin (μU/ml)×fasting plasma glucose (mg/dL)/405; and HOMA-β adopts the following formula to assess β-cell function: (20×fasting plasma insulin (μU/ml))/(fasting plasma glucose (mg/dL)−63). For adults, the cutoff value indicating IR is a HOMA-IR value of 2.5; and the cutoff value indicating a decline of β-cell function is a HOMA-β value of 66%. In some embodiments, HOMA-IR and HOMA-β values are calculated for subjects in a third database, i.e., the combined database of a first database and a second database; and the patients or participants are separated into non-IR group, IR group, non-β-cell deficiency group, β-cell deficiency group, according to the corresponding HOMA-IR value (≤ or >2.5) and HOMA-β value (<66%).

In at least one embodiment of the present disclosure, the database includes a first database and a second database. The first database and the second database are derived from a first population of the subject and a second population of the subject, respectively. In some embodiments, the race of the first population is different from the race of the second population. For instance, the first population is non-Asian, and the second population is Asian, but the present disclosure is not limited thereto.

### Methodology

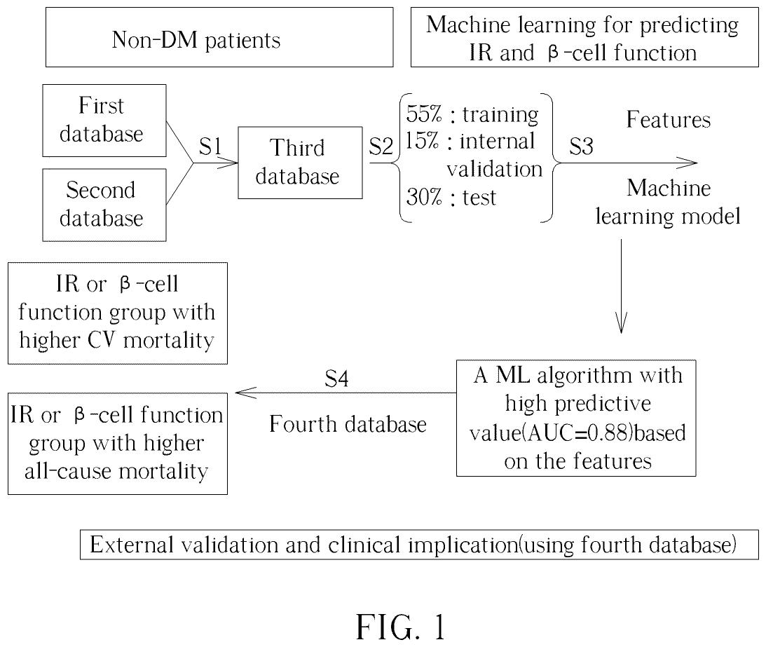

Referring to FIG. 1, system and method for predicting insulin resistance and/or pancreatic β-cell function is illustrated, and the operation steps are denoted as arrows and explained herefrom. Specifically, the term “ML” shown in FIG. 1 is an abbreviation of machine learning.

### Study Protocol and Participants of the Present Disclosure

In some embodiments, a first data set from a first database (e.g., National Health and Nutrition Examination Survey (NHANES) database), a second data set from a second database (e.g., MAJOR (MJ) research database), and a fourth data set from a fourth database (e.g., Taiwan biobank database (TWB)) are used in the present disclosure.

In some embodiments, a first database is the NHANES database, and the NHANES database is a health-related program in the USA. The health survey of the health-related program is launched periodically by the Centers for Disease Control (CDC) and Prevention's National Center for Health Statistics (NCHS). The first data is released to the public for research free of charge. The Research Ethics Review Board at the NCHS approved the study of the present disclosure, and all participants or proxies provided written informed consent. Examinations included anthropometric measurements, questionnaires on health and nutrition, and laboratory tests. Participants completed the questionnaires during in-home interviews. The participants in the NHANES from Jan. 1, 1999 to Dec. 31, 2012 are analyzed in the present disclosure. Participants are excluded from the analyses if they are aged <18 years old, have incomplete laboratory data, or have DM.

In some embodiments, a second database is the MJ research database, and the MJ research database is the most detailed and accurate medical database in Taiwan (R.O.C.). The Taiwan MJ Cohort resource is an ongoing and dynamic prospective study of women and men who have participated in a large health examination program run by the MJ Health Management Institution, Taiwan (www.mjclinic.com. tw). The Taiwan MJ group is the largest health management group in Asia. Details of the Taiwan MJ Cohort study population and data collection methods have been reported elsewhere. This MJ Cohort has enrolled approximately 600,000 Taiwanese individuals since 1994. Participants complete a standardized protocol including a health history questionnaire, multi-phasic blood panel, and laboratory tests, such as lung function, cardiogram, urinalysis and stool tests. These procedures conform to the International Organization for Standardization 9001 for quality management. After the first examination, all participants were encouraged to return annually, and all data are updated by the MJ Health Research Foundation.

In some embodiments, step S1 denotes that a database combining module will merge the first database and the second database into a third database, i.e., a third database is the combination of a first database and a second database.

In some embodiments, a fourth database is the Taiwan Biobank (TWB) database, and TWB project is launched by Taiwan's Ministry of Health and Welfare launched the TWB project for collecting national genetic and laboratory data of Taiwanese people. Taiwan has one of the most detailed health databases in the world, covering up to 99% of its population. TWB is a rich biomedical research database. There are currently 174,077 participants, 84,276,467 questionnaires, 72,076,086 data on body weight/height, and 79,314,928 laboratory data in the TWB database. Biomarkers and genetic data are generated for all participants from urinary and blood samples. The participants in the TWB population from 10 Dec. 2008 to 30 Nov. 2018 are analyzed in the present disclosure.

In some embodiments, the fourth database of the present disclosure, e.g. the TWB database, is used for external validation to establish whether estimated IR or β-cell function from the third database can predict mortality in nondiabetic individuals via applying a diagnostic algorithm.

In some embodiments, participants are collected from the first database, the second database, and the fourth databases, e.g., the NHANES database (1999-2012), the MJ database (2008-2017), and the TWB database (10 Oct. 2008 to 30 Nov. 2018), respectively.

The baseline variables include age (years old), gender, body mass index (BMI) (weight in kg divided by height² in meters), HOMA-IR (except for TWB database), total cholesterol (TC) (mg/dL), high-density lipoprotein (HDL) (mg/dL), triglyceride (mg/dL), FPG (mg/dL), and glycohemoglobin (HbAlc) (%). DM is defined according to the guideline of DM of the American Diabetes Association (ADA).

In some embodiments, a third feature set of the third database of the present disclosure is used for machine learning to achieve maximal utilization of IR or β-cell function. The third feature set is well known as factors related to IR or β-cell function in the art.

### A Machine Learning Model Building Process of the Present Disclosure

In some embodiments, step S2 denotes that the 55% of the patient population in the third database of the present disclosure is randomly selected as the training group for model building, 15% as the internal validation group for hyperparameter optimization and independent 30% for the test group.

In some embodiments, 78.6% (55/70) of the 70% patient population (i.e., the training group (55%) and the internal validation group (15%)) in the third database of the present disclosure is spat around for the training predictive model of the machine learning model of the present disclosure to avoid overfitting. The ratio (78.6%) was generally lower than in other predictive models (80%). The ratio (21.4%) (15/70) for internal validation of the performance is generally higher than that of other predictive models (20%), indicating the algorithm of the present disclosure has more patients for validation of the model performance.

In some embodiments, the Synthetic Minority Oversampling Technique (SMOTE) preprocessing algorithm is used for sample-balance between the target and non-target populations in the training group. In some embodiments, step S3 denotes that the third feature set of the present disclosure used to predict IR (HOMA-IR>2.5) is easily accessible variables, and the variables are, but not limited to, age, gender, race, body mass index (BMI), fasting plasma/blood glucose (fg/FBG/FPG), glycohemoglobin (HbAlc), triglyceride (tg), total cholesterol (TC/chol), and/or high-density lipoprotein (HDL) cholesterol (HDL-cholesterol/hdlc). In some embodiments, step S3 denotes that the third feature set of the present disclosure used to predict β-cell function (HOMA-β value <66%) is easily accessible variables, and the variables are, but not limited to, age, gender, race, body mass index (BMI), fasting plasma/blood glucose (fg/FBG/FPG), triglyceride (tg), total cholesterol (TC/chol), high-density lipoprotein (HDL) cholesterol (HDL-cholesterol/hdlc), glutamate oxaloactate transaminase (GOT), glutamate pyruvate transaminase (GPT), waist circumference (WC), total bilirubin (TB), albumin (alb), systolic blood pressure (sbp), diastolic blood pressure (dbp), estimated glomerular filtration rate (eGFR), and/or creatinine (CRE).

In some embodiments, first, a deep neural network (DNN) was used to estimate the first chosen method. Other traditional methods of machine learning algorithm, such as random forests (RFs), eXtreme Gradient Boosting (XGboost), and logistic regression algorithms are also used. Their accuracies were then compared with the logistic regression model. After completing the model training, another group is applied for testing. In the training group, curves of ROC (receiver operating characteristic) from different algorithms are compared. Both the ROC curve and AUC (area under curve) are used to compare classification performances of different classifiers. The targeted value of AUC (>0.80) suggested that the model of the present disclosure is adequate for predicting IR and/or β-cell function. In DNN, the entire structure of the deep neural network is designed as follows: 9 input layers→18 middle hidden layers→27 middle hidden layers→36 middle hidden layers→one dimensional output layer.

In some embodiments, the binary outcome of insulin resistance or β-cell function is set as the output layer. To avoid overfitting during the model training of the deep learning, we added a dropout layer between the hidden layers. The dropout rate was set at 0.2. As activation functions, scaled exponential linear units in the middle layer and hard sigmoid units in the output layer are employed. In the hyperparameter tuning process of XGboost and RFs, grid search is used to identify the optimal values on potential value combinations of the parameters.

In some embodiments, the Gini index is used to calculate the feature importance, i.e., the importance of each the variable of the third feature set of the present disclosure. For model explanations, SHAP values (SHapley Additive exPlanations) is used to explain how different machine learning models work. Finally, since IR and β-cell function are reported to predict mortality in nondiabetic individuals, the predictive value of mortality is also examined in IR prediction algorithm and β-cell-function prediction algorithm of the present disclosure.

In some embodiments, step S4 denotes that once good diagnostic performance of the aforementioned models has been achieved, the best model is applied to the fourth database (e.g. TWB database) for external validation, to determine its possible clinical implications (e.g. cardiovascular disease (CV) mortality and all-cause mortality).

### Clinical Implications of IR and β-Cell-Function: CV Mortality and All-Cause Mortality

Clinical implications of IR and β-cell-function are analyzed using the aforementioned trained model showing the highest predictive power. The information of mortality was derived through data linkages to active follow-up surveys and death certifications. According to the International Classification of Diseases (ICD) 9th or 10th revision, all-cause mortality is coded as ICD-9 0001-E999 and ICD-10 A00-Y98. CV mortality is coded as CV disease (ICD-9 390-456, and ICD-10 I00-I99), including coronary heart diseases (ICD-9 140-414, and ICD-10 I20-I25), stroke (ICD-9 430-438 and ICD-10 I60-I69), ischemic stroke (ICD-9 434 and ICD-10 163), and hemorrhagic stroke (ICD-9 4330-432, and ICD-10 I60-I62).

In some embodiments, a fourth database of the present disclosure is configured to provide a fourth data set of the present disclosure and used for external validation and evaluation of a clinical implication. In some embodiments, the clinical implication comprises cardiovascular mortality and all-cause mortality.

### Statistical Analysis of the Present Disclosure

The NHANES is a multiple and complex survey. To represent sample-weighted data, weighted data need to be calculated according to analytical guidelines (US NHANES: Analytical Guidelines, 2011-2014 and 2015-2016. Available online). However, original unweighted data from the NHANES and MJ databases is used to perform model building of machine learning and deep learning of the present disclosure. There are two reasons that the weighted data is not used in the present disclosure. First, weighting data is typically used to estimate nationwide occurrence rates/prevalence rates. There is no need to estimate the nationwide prevalence rate, and only the relationship between IR/β-cell-function and the third feature set of the present disclosure among individuals is needed to train the aforementioned model. Second, MJ database does not provide corresponding weights, and merging the data from the NHANES and MJ database is needed. Therefore, NHANES also used unweighted data. For unweighted data in the NHANES, MJ, and TWB databases, continuous variables are reported as means±standard deviation (SD) and categorical data as numbers (percentages). Differences in clinical variables between IR/β-cell-function statuses are assessed using the Chi-square test for categorical variables, or independent t-test for continuous variables. Univariate and multivariate logistic regression with restricted cubic spline approach is used to identify the non-linear relationship between selected features and IR/β-cell-function which could be used to compare the pattern of associations between machine learning and traditional statistical methods.

In some embodiments, a feature extraction module of the present disclosure collects and processes a data set of the present disclosure by deriving at least one of mean, standard deviation of the mean, number, percentage, coefficient of variance, and slope and R square of linear regression as a variable of a feature set of the present disclosure for building a machine learning model of the present disclosure.

In some embodiments, a fourth data set from the TWB database is applied as input to the aforementioned algorithms and the output is the predicted probability of IR/β-cell-function for each participant. Predicted probability greater than 0.5 is defined as the cutoff point for belonging to the IR group or β-cell-deficiency group. The Kaplan-Meier survival curve with Log-Rank and proportional HR model for time-to-event analysis test are used to compare the CV or all-cause mortality between predicted IR, non-IR, β-cell-deficiency, non-β-cell-deficiency groups. All reported p-values are two-sided and considered significant at p<0.05. Deep learning algorithms and other ML (including XGBoost, RFs and DNN) are conducted in Keras (version 2.4.0), TensorFlow (version 1.10.0) and Python (version 3.6.5). Univariate and multivariate analyses for CV and all-cause mortality are also performed. We also compared the predictive powers of the algorithm to Framingham score by C index and misclassification statistics. All statistical analyses are performed using SAS for Windows (version 9.4; SAS, Cary, NC). The present disclosure is approved by the Ethics Committee of Taichung Veterans General Hospital, IRB number: CE20023A. All procedures are performed in accordance with the relevant guidelines and regulations.

### Results

Participant Selections from the NHANES, MJ, and TWB Databases

Initially, 71,916 participants from the NHANES database (1999-2012) and 14,359 participants from the MJ database (2008-2017) are included in the present disclosure. After exclusion, there are 25,737 participants (14,211 from the NHANES and 12,526 from the MJ database) in the final analysis. Excluded participants are as follows: 26,373 from the NHANES database, and 72 from the MJ database due to age <18 years; 28,939 from the NHANES and 657 from the MJ database due to incomplete laboratory data; 2393 from the NHANES and 1104 from the MJ database due to DM history. Of all participants in the third database of the present disclosure (i.e. combined database of NHANES and MJ databases), 14,705 participants are randomly selected to serve as the training group, and 4018 participants who served as the internal validation group. In the training group, the model is trained with the following algorithms: XGBoost, RFs, logistic regression and DNN, and their AUCs are finally compared. A total of 8014 participants are included in the test group for model evaluation. Participants from the TWB database are further collected from 10 Dec. 2008 to 30 Nov. 2018 for external validation and the evaluation of clinical implication.

Baseline Characteristics of Participants from Caucasian (NHANES) and Asian Database (MJ in Taiwan)

The first database and the second database of the present disclosure (NHANES vs. MJ) include two races, i.e., Caucasian and Asian populations, respectively; however, age (44.84 vs. 45.06 y/o) and gender rates (48.29 vs. 50.44%) are similar. In addition, fasting blood/plasma glucose (91.54 vs. 100.07 mg/dL), glycohemoglobin (5.32 vs. 5.2%), and total cholesterol (198.45 vs. 199.13 mg/dL) were also similar. However, insulin resistance is much higher in patients from NHANES than from MJ (2.84 vs. 1.92). Furthermore, individuals from NHANES had higher levels of plasma insulin (12.1 vs. 7.67 mIU/L), BMI (27.86 vs. 23.78 kg/m2), and triglycerides (126.21 vs. 116.59 mg/dL).

Baseline Characteristics of Participants with or without Diabetes Mellitus (DM) According to HOMA-IR (≤2.5 or >2.5) and/or HOMA-β (<66%)

Regarding the three groups: training, internal validation and test groups of the present disclosure, there is no statistically significant difference among the three groups (training, internal validation, and test groups). Relevant details according to insulin resistance or not from all participants of the first database, second database, and third database are shown in Table 1. Baseline characteristics are similar among participants from the NHANES and MJ databases, except in the NHANES participants, there is a higher mean HOMA-IR value (2.84 vs. 1.92), higher fasting plasma insulin level (12.1 vs. 7.67 mIU/L), higher BMI (27.86 vs. 23.78 kg/m2), higher triglyceride (126.21 vs. 116.59 mg/dL), lower FPG (91.54 vs. 100.07 mg/dL), and lower HDL-cholesterol (53.88 vs. 57.77 mg/dL). In the combined NHANES and MJ databases (i.e. the third database of the present disclosure), the IR group (IR>2.5) is significantly (p<0.0001) older (46.06±16.58 vs. 45.28±15.34), had a higher proportion of males (54.3 vs. 46.5%), had higher HOMA-IR (4.53±2.94 vs. 1.47±0.53), higher plasma insulin level (17.84±10.88 vs. 6.17±2.17), higher BMI (30.05±6.05 vs. 24±4.03), higher HbAlc (5.43±0.41 vs. 5.21±0.41), higher FPG (99.08±10.62 vs. 93.92±9.78), lower HDL-cholesterol (49.19±12.97 vs. 59.2±15.43), higher TC (201.35±39.97 vs. 197.16±38.17) and higher triglyceride (155.51±112.06 vs. 104.25±78.04).

Further, relevant details according to pancreatic β-cell function from all participants of the first, second, and third databases are shown in Table 2.

TABLE 1
Baseline data according to insulin resistance or not from all participants of the
first database, the second database and the third database of the present disclosure,
i.e., NHANES, MJ, and combined NHANES and MJ database, respectively.

| | Overall | | HOMA-IR ≤ 2.5 | | HOMA-IR >2.5 | | p-value |
|---|---|---|---|---|---|---|---|
| | NHANES database | | | | | | |
| N | 14211 | | 8082 | | 6129 | | |
| Age (years old) | 44.84 | (44.29-45.4) | 44.23 | (43.58-44.88) | 45.73 | (45.12-46.34) | <0.0001 |
| Male, n (%) | 6802 | (48.29) | 3730 | (44.98) | 3072 | (53.12) | <0.0001 |
| Female, n (%) | 7409 | (51.71) | 4352 | (55.02) | 3057 | (46.88) | <0.0001 |
| HOMA-IR | 2.84 | (2.77-2.91) | 1.48 | (1.46-1.5) | 4.83 | (4.7-4.96) | <0.0001 |
| Plasma insulin | 12.1ma ins | | 6.51ma in | | 19.46a insu | | <0.0001 |
| level (mIU/L) | | | | | | | |
| Body Mass | 27.86 | (27.72-28) | 25.39 | (25.26-25.52) | 31.45 | (31.22-31.69) | <0.0001 |
| Index (kg/m²) | | | | | | | |
| Glycohemoglobin: (%) | 5.32 | (5.31-5.34) | 5.25 | (5.23-5.27) | 5.43 | (5.41-5.44) | <0.0001 |
| Fasting plasma | 91.54 | (91.22-91.86) | 88.27 | (87.93-88.6) | 96.3 | (95.92-96.68) | <0.0001 |
| glucose, mg/dl | | | | | | | |
| HDL-cholesterol | 53.88 | (53.47-54.3) | 58.03 | (57.52-58.54) | 47.84 | (47.39-48.29) | <0.0001 |
| (mg/dL) | | | | | | | |
| Cholesterol | 198.45 | (197.36-199.53) | 196.53 | (195.25-197.82) | 201.23 | (199.71-202.76) | <0.0001 |
| (mg/dL) | | | | | | | |
| Triglycerides | 126.21 | (123.6-128.83) | 104.22 | (101.12-107.32) | 158.25 | (153.55-162.96) | <0.0001 |
| (mg/dL) | | | | | | | |
| | MJ database | | | | | | |
| N | 12526 | | 9824 | | 2702 | | |
| Age | 45.06 ± 11.49 | | 45.14 ± 11.55 | | 44.73 ± 11.26 | | <0.0001 |
| Male, n (%) | 6318 | (50.44) | 4595 | (46.77) | 1723 | (63.77) | <0.0001 |
| Female, n (%) | 6208 | (49.56) | 5229 | (53.23) | 979 | (36.23) | <0.0001 |
| HOMA-IR | 1.92 ± 1.26 | | 1.44 ± 0.51 | | 3.68 ± 1.58 | | <0.0001 |
| Plasma insulin | 7.67ma in | | 5.88ma in | | 14.14a ins | | <0.0001 |
| level (mIU/L) | | | | | | | |
| Body Mass | 23.78 ± 3.76 | | 22.81 ± 3.09 | | 27.3 ± 3.89 | | <0.0001 |
| Index (kg/m²) | | | | | | | |
| Glycohemoglobin: (%) | 5.2 ± 0.44 | | 5.16 ± 0.43 | | 5.38 ± 0.44 | | <0.0001 |
| Fasting plasma | 100.07 ± 8.3 | | 98.57 ± 7.72 | | 105.49 ± 8.08 | | <0.0001 |
| glucose, mg/dl | | | | | | | |
| HDL-cholesterol | 57.77 ± 14.52 | | 59.93 ± 14.66 | | 49.9 ± 10.8 | | <0.0001 |
| (mg/dL) | | | | | | | |
| Cholesterol | 199.13 ± 35.28 | | 197.77 ± 34.9 | | 204.09 ± 36.18 | | <0.0001 |
| (mg/dL) | | | | | | | |
| Triglycerides | 116.59 ± 88.9 | | 104.01 ± 73.04 | | 162.33 ± 120.74 | | <0.0001 |
| (mg/dL) | | | | | | | |
| | Combined NHANES and MJ database | | | | | | |
| N | 26737 | | 17906 | | 8831 | | |
| Age | 45.54 ± 15.76 | | 45.28 ± 15.34 | | 46.06 ± 16.58 | | <0.0001 |
| Male, n (%) | 13120 | (49.07) | 8325 | (46.49) | 4795 | (54.3) | <0.0001 |
| Female, n (%) | 13617 | (50.93) | 9581 | (53.51) | 4036 | (45.7) | <0.0001 |

| | | | | |
|---|---|---|---|---|
| HOMA-IR | 2.48 ± 2.26 | 1.47 ± 0.53 | 4.53 ± 2.94 | <0.0001 |
| Plasma insulin | 10.02 ± 8.51 | 6.172 ± 8.5 | 17.84 ± 8.518 | <0.0001 |
| level (mIU/L) | | | | |
| Body Mass Index | 26 ± 5.57 | 24 ± 4.03 | 30.05 ± 6.05 | <0.0001 |
| (kg/m**2) | | | | |
| Glycohemoglobin: (%) | 5.28 ± 0.42 | 5.21 ± 0.41 | 5.43 ± 0.41 | <0.0001 |
| Fasting plasma | 95.62 ± 10.35 | 93.92 ± 9.78 | 99.08 ± 10.62 | <0.0001 |
| glucose, mg/dl | | | | |
| HDL-cholesterol | 55.89 ± 15.4 | 59.2 ± 15.43 | 49.19 ± 12.97 | <0.0001 |
| (mg/dL) | | | | |
| Cholesterol | 198.54 ± 38.82 | 197.16 ± 38.17 | 201.35 ± 39.97 | <0.0001 |
| (mg/dL) | | | | |
| Triglycerides | 121.18 ± 93.85 | 104.25 ± 78.04 | 155.51 ± 112.06 | <0.0001 |
| (mg/dL) | | | | |

TABLE 2
Baseline data according to pancreatic B-cell function or not from all participants
of the first database, the second database and the third database of the present
disclosure, i.e., NHANES, MJ, and combined NHANES and MJ database, respectively.

| | | | |
|---|---|---|---|
| overall | Homa-beta >66 | Homa-beta <=66 | p-value |

NHANES database

| N | 11792 | | 9065 | | 2727 | | |
|---|---|---|---|---|---|---|---|
| Homa-beta | 139.39 ± 267.86 | | 167.05 ± 299.96 | | 47.45 ± 13.06 | | <.0001 |
| Age (years old) | 44.6 ± 18.99 | | 43.07 ± 18.65 | | 49.68 ± 19.23 | | <.0001 |
| waist circumference (cm) | 95.58 ± 15.25 | | 97.89 ± 15.47 | | 87.9 ± 11.52 | | <.0001 |
| BMI (kg/m²) | 27.83 ± 6.09 | | 28.87 ± 6.22 | | 24.39 ± 4.04 | | <.0001 |
| Systolic blood pressure | 121.65 ± 18.5 | | 121.33 ± 17.99 | | 122.72 ± 20.05 | | 0.0012 |
| (mmHg) | | | | | | | |
| Diastolic blood pressure | 68.9 ± 13.01 | | 69.15 ± 12.9 | | 68.08 ± 13.33 | | 0.0002 |
| (mmHg) | | | | | | | |
| Fasting plasma glucose, | 96.6 ± 10.25 | | 96.09 ± 10.33 | | 98.3 ± 9.77 | | <.0001 |
| mg/dl | | | | | | | |
| HDL-cholesterol (mg/dL) | 54.39 ± 15.81 | | 52.52 ± 14.92 | | 60.62 ± 17.05 | | <.0001 |
| Cholesterol (mg/dL) | 195.73 ± 42.13 | | 195.5 ± 42.05 | | 196.5 ± 42.42 | | 0.28 |
| Triglycerides (mg/dL) | 121.24 ± 94.11 | | 129.02 ± 99.96 | | 95.35 ± 64.89 | | <.0001 |
| eGFR (mL/min/1.73 m²) | 101.84 ± 24.47 | | 103.89 ± 24.53 | | 95.04 ± 22.98 | | <.0001 |
| Creatinine (mg/dL) | 0.84 ± 0.36 | | 0.82 ± 0.33 | | 0.9 ± 0.45 | | <.0001 |
| Albumin (g/dL) | 4.26 ± 0.37 | | 4.24 ± 0.38 | | 4.3 ± 0.33 | | <.0001 |
| Total bilirubin (mg/dL) | 0.75 ± 0.33 | | 0.72 ± 0.3 | | 0.84 ± 0.4 | | <.0001 |
| AST (Aspartate | 25.68 ± 24.92 | | 25.56 ± 21.28 | | 26.1 ± 34.34 | | 0.3236 |
| Aminotransferase) | | | | | | | |
| ALT (Alanine | 25.38 ± 28.7 | | 26.15 ± 24.35 | | 22.83 ± 39.8 | | <.0001 |
| Aminotransferase) | | | | | | | |
| male, n (%) | 5728 | (48.58) | 4157 | (45.86) | 1571 | (57.61) | <.0001 |
| female, n (%) | 6064 | (51.42) | 4908 | (54.14) | 1156 | (42.39) | |

MJ database

| N | 13005 | | 6427 | | 6578 | | |
|---|---|---|---|---|---|---|---|
| Homa-beta | 75.07 ± 44.96 | | 103.11 ± 49 | | 47.68 ± 11.5 | | <.0001 |
| Age (years old) | 45.09 ± 11.52 | | 43.12 ± 11.14 | | 47.01 ± 11.56 | | <.0001 |
| waist circumference (cm) | 78.63 ± 10.43 | | 82.1 ± 10.77 | | 75.25 ± 8.86 | | <.0001 |
| BMI (kg/m²) | 23.8 ± 3.79 | | 25.23 ± 4.04 | | 22.41 ± 2.91 | | <.0001 |
| Systolic blood pressure | 115.82 ± 16.93 | | 117.75 ± 16.88 | | 113.93 ± 16.77 | | <.0001 |
| (mmHg) | | | | | | | |
| Diastolic blood pressure | 73.98 ± 11.16 | | 75.24 ± 11.4 | | 72.75 ± 10.77 | | <.0001 |
| (mmHg) | | | | | | | |
| Fasting plasma glucose, | 100.17 ± 8.37 | | 99.53 ± 8.33 | | 100.79 ± 8.36 | | <.0001 |
| mg/dl | | | | | | | |
| HDL-cholesterol (mg/dL) | 57.74 ± 14.5 | | 54.1 ± 13 | | 61.3 ± 14.99 | | <.0001 |
| Cholesterol (mg/dL) | 199.1 ± 35.14 | | 199.75 ± 35.06 | | 198.48 ± 35.21 | | 0.039 |
| Triglycerides (mg/dL) | 116.85 ± 88.57 | | 135.42 ± 93.8 | | 98.71 ± 79.05 | | <.0001 |
| eGFR (mL/min/1.73 m²) | 81.12 ± 12.54 | | 81.72 ± 12.87 | | 80.54 ± 12.19 | | <.0001 |
| Creatinine (mg/dL) | 0.96 ± 0.22 | | 0.97 ± 0.25 | | 0.95 ± 0.19 | | <.0001 |
| Albumin (g/dL) | 4.41 ± 0.2 | | 4.42 ± 0.2 | | 4.39 ± 0.2 | | <.0001 |
| Total bilirubin (mg/dL) | 0.99 ± 0.38 | | 0.93 ± 0.36 | | 1.06 ± 0.39 | | <.0001 |
| AST (Aspartate | 23.99 ± 11.05 | | 25.23 ± 13 | | 22.78 ± 8.57 | | <.0001 |
| Aminotransferase) | | | | | | | |
| ALT (Alanine | 28.54 ± 22.57 | | 33.95 ± 28 | | 23.24 ± 13.57 | | <.0001 |
| Aminotransferase) | | | | | | | |
| male, n (%) | 6585 | (50.63) | 3393 | (52.79) | 3192 | (48.53) | <.0001 |
| female, n (%) | 6420 | (49.37) | 3034 | (47.21) | 3386 | (51.47) | |

Combined NHANES and MJ database

| N | 24797 | | 15492 | | 9305 | | |
|---|---|---|---|---|---|---|---|
| Homa-beta | 105.66 ± 190.29 | | 140.52 ± 233.74 | | 47.61 ± 11.98 | | <.0001 |
| Age (years old) | 44.85 ± 15.53 | | 43.09 ± 15.97 | | 47.79 ± 14.29 | | <.0001 |
| waist circumference (cm) | 86.69 ± 15.47 | | 91.34 ± 15.77 | | 78.96 ± 11.3 | | <.0001 |
| BMI (kg/m²) | 25.72 ± 5.4 | | 27.36 ± 5.71 | | 22.99 ± 3.4 | | <.0001 |
| Systolic blood pressure | 118.59 ± 17.93 | | 119.84 ± 17.63 | | 116.51 ± 18.23 | | <.0001 |
| (mmHg) | | | | | | | |
| Diastolic blood pressure | 71.56 ± 12.34 | | 71.67 ± 12.66 | | 71.38 ± 11.77 | | 0.0644 |
| (mmHg) | | | | | | | |
| Fasting plasma glucose, | 98.47 ± 9.48 | | 97.52 ± 9.7 | | 100.06 ± 8.87 | | <.0001 |
| mg/dl | | | | | | | |
| HDL-cholesterol (mg/dL) | 56.15 ± 15.23 | | 53.18 ± 14.17 | | 61.1 ± 15.63 | | <.0001 |
| Cholesterol (mg/dL) | 197.5 ± 38.66 | | 197.26 ± 39.36 | | 197.9 ± 37.48 | | 0.2066 |
| Triglycerides (mg/dL) | 118.94 ± 91.27 | | 131.67 ± 97.5 | | 97.73 ± 75.19 | | <.0001 |
| eGFR (mL/min/1.73 m²) | 90.98 ± 21.78 | | 94.69 ± 23.24 | | 84.79 ± 17.42 | | <.0001 |
| Creatinine (mg/dL) | 0.9 ± 0.3 | | 0.88 ± 0.3 | | 0.93 ± 0.29 | | <.0001 |
| Albumin (g/dL) | 4.33 ± 0.3 | | 4.32 ± 0.33 | | 4.36 ± 0.25 | | <.0001 |
| Total bilirubin (mg/dL) | 0.87 ± 0.38 | | 0.8 ± 0.35 | | 0.99 ± 0.4 | | <.0001 |
| AST (Aspartate | 24.8 ± 18.97 | | 25.42 ± 18.31 | | 23.75 ± 19.99 | | <.0001 |
| Aminotransferase) | | | | | | | |
| ALT (Alanine | 27.03 ± 25.72 | | 29.38 ± 26.21 | | 23.12 ± 24.38 | | <.0001 |
| Aminotransferase) | | | | | | | |
| male, n (%) | 12313 | (49.66) | 7550 | (48.73) | 4763 | (51.19) | 0.0002 |
| female, n (%) | 12484 | (50.34) | 7942 | (51.27) | 4542 | (48.81) | |

### Baseline Data of the TWB Database for External Validation and the Evaluation of Clinical Implications

In some embodiments, a fourth data set from the fourth database of the present disclosure, e.g., TWB database, are fed into the XGBoost algorithm to be separated into predicted non-IR (n=86, 283) and IR groups (8774). Participants with predicted IR is significantly (p<0.0001) younger (47.51±10.81 vs. 49.42±10.86 y/o), had a higher proportion of males (48.80 vs. 33.39%), and have higher BMI (30.27±3.54 vs. 23.32±2.97 kg/m2), higher HbAlc (5.83±0.32 vs. 5.57±0.33%), higher FPG (99.35±9.44 vs. 90.98±6.99 mg/dL), lower HDL-cholesterol (43.39±8.59 vs. 56.32±13.25 mg/dL), higher TC (200.22±35.75 vs. 195.44±34.98 mg/dL), and higher triglyceride (201.95±150.76 vs. 100.54±67.6 mg/dL).

Predictive Values from Machine Learning Models

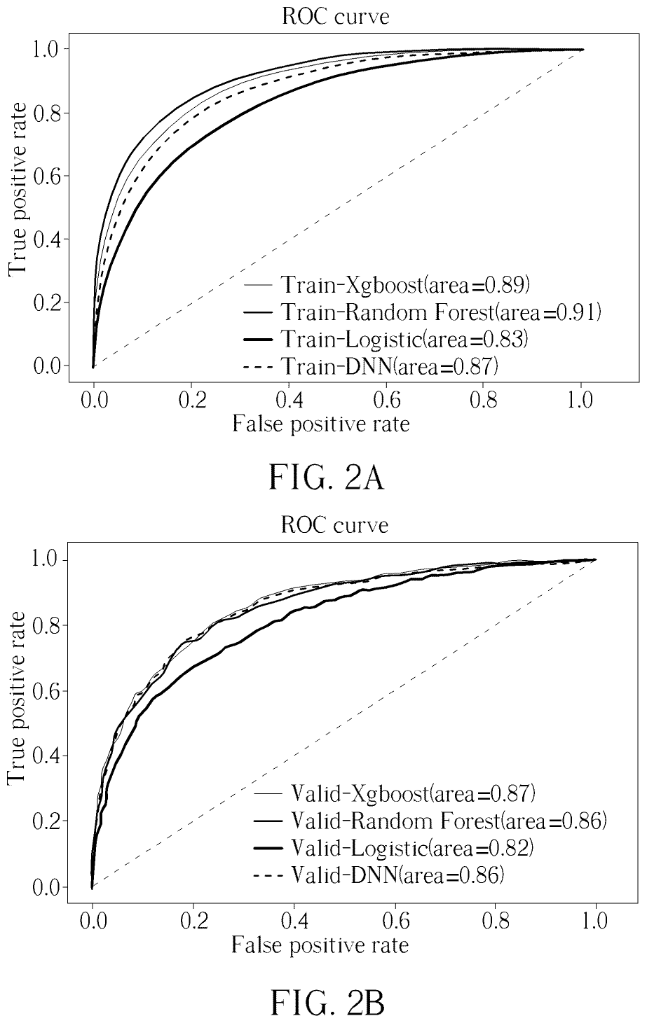

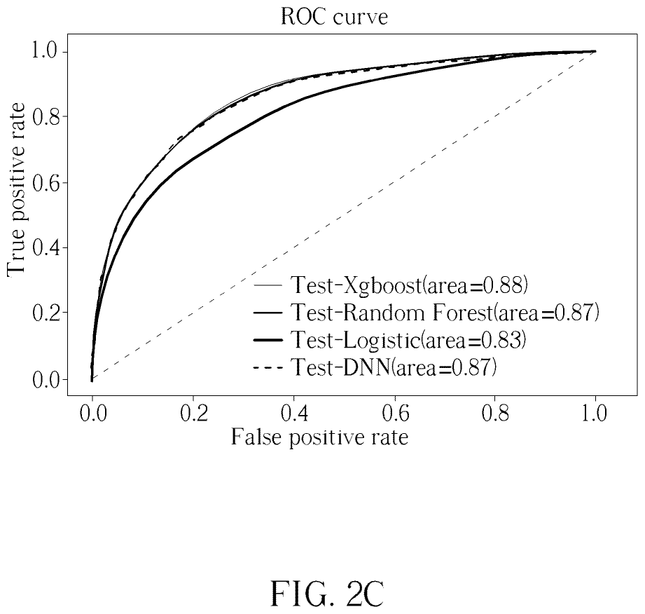

The results of the internal validation group and test group using the algorithms of XGboost, RFs, logistic regression and DNN are shown in Table 3. FIG. 2A shows the ROC curve in the training group. FIG. 2B shows that AUC of ROC are all >0.8 (highest was XGboost, 0.87) in the internal validation group. In the internal validation group, XGboost also has the highest accuracy (0.80), sensitivity (0.63), specificity (0.89), positive predictive value (PPV) (0.74) and negative predictive value (NPV) (0.8) according to Table 3. FIG. 2C shows that all AUC of ROC are also >0.80 (highest being XGboost, 0.88) in the test group. In addition, XGboost also has the highest accuracy (0.81), sensitivity (0.64) and NPV (0.83), and RFs have the highest specificity (0.90) and PPV (0.75) in the test group according to Table 3.

TABLE 3
The area under curve (AUC) of the receiver operating
characteristic (ROC) curve, accuracy, sensitivity,
specificity, positive predictive value (PPV) , and
negative predictive value (NPV) from different models.

| | | | |
|---|---|---|---|
| | Random | | Deep neural |
| | forests | Logistic | Network |
| XGboost | (RFs) | regression | (DNN) |

Internal Validation group

| | | | | |
|---|---|---|---|---|
| AUC of ROC | 0.87 | 0.86 | 0.82 | 0.86 |
| Accuracy | 0.80 | 0.80 | 0.78 | 0.80 |
| Sensitivity | 0.63 | 0.60 | 0.56 | 0.61 |
| Specificity | 0.89 | 0.89 | 0.89 | 0.89 |
| PPV | 0.74 | 0.74 | 0.71 | 0.74 |
| NPV | 0.83 | 0.82 | 0.80 | 0.82 |

Test group

| | | | | |
|---|---|---|---|---|
| AUC of ROC | 0.88 | 0.87 | 0.83 | 0.87 |
| Accuracy | 0.81 | 0.81 | 0.78 | 0.81 |
| Sensitivity | 0.64 | 0.62 | 0.56 | 0.64 |
| Specificity | 0.89 | 0.90 | 0.88 | 0.89 |
| PPV | 0.74 | 0.75 | 0.70 | 0.75 |
| NPV | 0.83 | 0.83 | 0.80 | 0.83 |

Comparisons of AUC among models

| | XGboost | XGboost vs. | XGboost vs. logistic |
|---|---|---|---|
| | vs. RFs | DNN | regression |
| Internal | 0.0007 | 0.0264 | <0.0001 |
| Validation | | | |
| group | | | |
| Test group | <0.0001 | 0.0022 | <0.0001 |

Table 4 and Table 5 show the AUC of the ROC curve of the machine learning model of the present disclosure for predicting IR and β-cell function, respectively, using different feature set of the present disclosure, illustrating the predictive power.

In some embodiments, the machine learning model of the present disclosure for predicting IR and β-cell function may be employed by the classification algorithms and additional algorithms, but the present disclosure is not limited thereto. The classification algorithms of some embodiments may be, but not limited to, XGBoost, RFs, logistic regression, and/or DNN. The additional algorithms of some embodiments may be, but not limited to, linear regression, polynomial regression, support vector regression, decision tree regression, random forest regression, ridge regression, lasso regression, elastic net regression, logistic regression, decision tree regression, gradient boosting, and/or K-Nearest regression.

In some embodiments, the true value of HOMA-IR and/or HOMA-β of the subject may be determined by the classification algorithms, the predicted probability of IR/β-cell-function of the subject, and additional algorithms; the classification algorithms of the present disclosure may be, but not limited to, XGBoost, RFs, logistic regression, and/or DNN; and the additional algorithms of the present disclosure may be, but not limited to, linear regression, polynomial regression, support vector regression, decision tree regression, random forest regression, ridge regression, lasso regression, elastic net regression, logistic regression, decision tree regression, gradient boosting, and/or K-Nearest regression.

In some embodiments, a model building and optimization module of the present disclosure builds a machine learning model of the present disclosure based on a feature set of the present disclosure to predict the insulin resistance and the pancreatic β-cell function of the non-diabetes mellitus patient by classifying the non-diabetes mellitus patient into an insulin resistance group, a non-insulin resistance group, β-cell deficiency group, and a non-β-cell deficiency group, and generating a corresponding predictive value and a classification performance value thereof.

In some embodiments, the classification performance value is an area under curve of a receiver operating characteristic curve of the machine learning model of the present disclosure.

TABLE 4
The area under curve (AUC) of the receiver operating characteristic
(ROC) curve from the machine learning model for predicting IR
(the symbol “N/A” indicates that the data is not shown).

| Condition | | | AUC of the |
|---|---|---|---|
| No. | Feature set | Race | ROC curve |
| 1-1 | age, gender, race, and BMI | Asian | 0.8293 |
| | | population | |
| 1-2 | age, gender, race, and BMI | Caucasian | 0.793 |
| | | population | |
| 2-1 | age, gender, race, and BMI | Asian | 0.865 (if the FPG is |
| | | population | selected) |
| | fasting plasma glucose (FPG) | | 0.8364 (if the HbA1c |
| | or glycohemoglobin (HbA1c) | | is selected) |
| 2-2 | age, gender, race, and BMI | Caucasian | 0.839 (if the FPG is |
| | | population | selected) |
| | fasting plasma glucose (FPG) | | 0.8041 (if the HbA1c |
| | or glycohemoglobin (HbA1c) | | is selected) |
| 3-1 | age, gender, race, BMI, and | Asian | 0.8463 |
| | triglyceride | population | |
| 3-2 | age, gender, race, BMI, and | Caucasian | 0.8175 |
| | triglyceride | population | |
| 4-1 | age, gender, race, BMI, and | Asian | 0.8471 (if the TC is |
| | triglyceride | population | selected) |
| | total cholesterol (TC) or | | 0.8518 (if the |
| | high-density lipoprotein | | HDL-cholesterol is |
| | (HDL) cholesterol | | selected) |
| 4-2 | age, gender, race, BMI, and | Caucasian | 0.8202 (if the TC is |
| | triglyceride | population | selected) |
| | total cholesterol (TC) or | | 0.8231 (if the HDL- |
| | high-density lipoprotein | | cholesterol is |
| | (HDL) cholesterol | | selected) |
| 5-1 | age, gender, race, and BMI | Asian | N/A |
| | liver function (GOT and GPT), | population | N/A |
| | total bilirubin (tb), and | | N/A |
| | albumin (alb) | | |
| 5-2 | age, gender, race, and BMI | Caucasian | N/A |
| | liver function (GOT and GPT), | population | N/A |
| | total bilirubin (tb), and | | N/A |
| | albumin (alb) | | |
| 6-1 | age, gender, race, and BMI | Asian | 0.8666 (if FPG and |
| | | population | TC are selected) |
| | fasting plasma glucose (FPG) | | 0.8736 (if FPG and |
| | or glycohemoglobin (HbA1c) | | HDL-cholesterol are |
| | | | selected) |
| | total cholesterol (TC) or | | 0.8369 (if HbA1c and |
| | high-density lipoprotein | | TC are selected) |
| | (HDL) cholesterol | | 0.8473 (if HbA1c and |
| | | | HDL-cholesterol are |
| | | | selected) |
| 6-2 | age, gender, race, and BMI | Caucasian | 0.8409 (if FPG and |
| | | population | TC are selected) |
| | fasting plasma glucose (FPG) | | 0.8509 (if FPG and |
| | or glycohemoglobin (HbA1c) | | HDL-cholesterol are |
| | | | selected) |
| | total cholesterol (TC) or | | 0.8041 (if HbA1c and |
| | high-density lipoprotein | | TC are selected) |
| | (HDL) cholesterol | | 0.8213 (if HbA1c and |
| | | | HDL-cholesterol are |
| | | | selected) |
| 7-1 | age, gender, race, BMI, and | Asian | N/A (if TC, GOT and |
| | fasting plasma glucose (FPG) | population | GPT are selected) |
| | | | N/A (if |
| | | | HDL-cholesterol, |
| | | | GOT and GPT are |
| | | | selected) |
| | total cholesterol (TC) or | | N/A (if TC and tb are |
| | high-density lipoprotein | | selected) |
| | (HDL) cholesterol | | N/A (if |
| | | | HDL-cholesterol and |
| | | | tb are selected) |
| | liver function (GOT and GPT), | | N/A (if TC and alb |
| | total bilirubin (tb), and | | are selected) |
| | albumin (alb) | | N/A (if |
| | | | HDL-cholesterol and |
| | | | alb are selected) |
| 7-2 | age, gender, race, BMI, and | Caucasian | N/A (if TC, GOT and |
| | fasting plasma glucose (FPG) | population | GPT are selected) |
| | | | N/A (if |
| | | | HDL-cholesterol, |
| | | | GOT and GPT are |
| | | | selected) |
| | total cholesterol (TC) or | | N/A (if TC and tb are |
| | high-density lipoprotein | | selected) |
| | (HDL) cholesterol | | N/A (if |
| | | | HDL-cholesterol and |
| | | | tb are selected) |
| | liver function (GOT and GPT), | | N/A (if TC and alb |
| | total bilirubin (tb), and | | are selected) |
| | albumin (alb) | | N/A (if |
| | | | HDL-cholesterol and |
| | | | alb are selected) |
| 8-1 | age, gender, race, BMI, | Asian | N/A (if TC, GOT and |
| | triglyceride and fasting | population | GPT are selected) |
| | plasma glucose (FPG) | | N/A (if |
| | | | HDL-cholesterol, |
| | | | GOT and GPT are |
| | | | selected) |
| | total cholesterol (TC) or | | N/A (if TC and tb are |
| | high-density lipoprotein | | selected) |
| | (HDL) cholesterol | | N/A (if |
| | | | HDL-cholesterol and |
| | | | tb are selected) |
| | liver function (GOT and GPT), | | N/A (if TC and alb |
| | | | are selected) |
| | total bilirubin (tb), and | | N/A (if |
| | albumin (alb) | | HDL-cholesterol and |
| | | | alb are selected) |
| 8-2 | age, gender, race, BMI, | Caucasian | N/A (if TC, GOT and |
| | triglyceride and fasting | population | GPT are selected) |
| | plasma glucose (FPG) | | N/A (if |
| | | | HDL-cholesterol, |
| | | | GOT and GPT are |
| | | | selected) |
| | total cholesterol (TC) or | | N/A (if TC and tb are |
| | high-density lipoprotein | | selected) |
| | (HDL) cholesterol | | N/A (if |
| | | | HDL-cholesterol and |
| | | | tb are selected) |
| | liver function (GOT and GPT), | | N/A (if TC and alb |
| | total bilirubin (tb), and | | are selected) |
| | albumin (alb) | | N/A (if |
| | | | HDL-cholesterol and |
| | | | alb are selected) |

TABLE 5
The area under curve (AUC) of the receiver operating characteristic
(ROC) curve from the machine learning model for predicting β-cell
function (the symbol “N/A” indicates that the data is not shown).

| Condition | | | AUC of the |
|---|---|---|---|
| No. | Feature set | Race | ROC curve |
| 1-1 | age, gender, race, and BMI | Asian | 0.7578 |
| | | population | |
| 1-2 | age, gender, race, and BMI | Caucasian | 0.7812 |
| | | population | |
| 2-1 | age, gender, race, and BMI | Asian | 0.7735 (if the FPG |
| | | population | is selected) |
| | fasting plasma glucose | | N/A (if the HbA1c is |
| | (FPG) or glycohemoglobin (HbA1c) | | selected) |
| 2-2 | age, gender, race, and BMI | Caucasian | 0.7928 (if the FPG |
| | | population | is selected) |
| | fasting plasma glucose | | N/A (if the HbA1c is |
| | (FPG) or | | selected) |
| | glycohemoglobin (HbA1c) | | |
| 3-1 | age, gender, race, BMI, and | Asian | 0.7812 |
| | triglyceride | population | |
| 3-2 | age, gender, race, BMI, and | Caucasian | 0.8034 |
| | triglyceride | population | |
| 4-1 | age, gender, race, BMI, and | Asian | 0.7823 (if the TC is |
| | triglyceride | population | selected) |
| | total cholesterol (TC) or | | 0.7833 (if the |
| | high-density lipoprotein | | HDL-cholesterol is |
| | (HDL) cholesterol | | selected) |
| 4-2 | age, gender, race, BMI, and | Caucasian | 0.8056 (if the TC is |
| | triglyceride | population | selected) |
| | total cholesterol (TC) or | | 0.8083 (if the |
| | high-density lipoprotein | | HDL-cholesterol is |
| | (HDL) cholesterol | | selected) |
| 5-1 | age, gender, race, and BMI | Asian | 0.7814 (if GOT and |
| | | population | GPT are selected) |
| | liver function (GOT and | | 0.7587 (if alb is |
| | GPT), total bilirubin (tb), | | selected) |
| | and albumin (alb) | | 0.763 (if tb is |
| | | | selected) |
| 5-2 | age, gender, race, and BMI | Caucasian | 0.7897 (if GOT and |
| | | population | GPT are selected) |
| | liver function (GOT and | | 0.7823 (if alb is |
| | GPT), total bilirubin (tb), | | selected) |
| | and albumin (alb) | | 0.7921 (if tb is |
| | | | selected) |
| 6-1 | age, gender, race, and BMI | Asian | 0.7736 (if FPG and |
| | | population | TC are selected) |
| | fasting plasma glucose | | 0.785 (if FPG and |
| | (FPG) or | | HDL-cholesterol |
| | glycohemoglobin (HbA1c) | | are selected) |
| | total cholesterol (TC) or | | N/A (if HbA1c and TC |
| | high-density lipoprotein | | are selected) |
| | (HDL) cholesterol | | N/A (if HbA1c and |
| | | | HDL-cholesterol |
| | | | are selected) |
| 6-2 | age, gender, race, and BMI | Caucasian | 0.7948 (if FPG and |
| | | population | TC are selected) |
| | fasting plasma glucose | | 0.8129 (if FPG and |
| | (FPG) or | | HDL-cholesterol |
| | glycohemoglobin (HbA1c) | | are selected) |
| | total cholesterol (TC) or | | N/A (if HbA1c and TC |
| | high-density lipoprotein | | are selected) |
| | (HDL) cholesterol | | N/A (if HbA1c and |
| | | | HDL-cholesterol |
| | | | are selected) |
| 7-1 | age, gender, race, BMI, and | Asian | 0.8024 (if TC, GOT |
| | fasting plasma glucose | population | and GPT are |
| | (FPG) | | selected) |
| | | | 0.8089 (if |
| | | | HDL-cholesterol, |
| | | | GOT and GPT are |
| | | | selected) |
| | total cholesterol (TC) or | | 0.7839 (if TC and tb |
| | high-density lipoprotein | | are selected) |
| | (HDL) cholesterol | | 0.7902 ( if |
| | | | HDL-cholesterol |
| | | | and tb are |
| | | | selected) |
| | liver function (GOT and | | 0.7787 (if TC and |
| | GPT), total bilirubin (tb), | | alb are selected) |
| | and albumin (alb) | | 0.7903 (if |
| | | | HDL-cholesterol |
| | | | and alb are |
| | | | selected) |
| 7-2 | age, gender, race, BMI, and | Caucasian | 0.8069 (if TC, GOT |
| | fasting plasma glucose | population | and GPT are |
| | (FPG) | | selected) |
| | | | 0.8189 (if |
| | | | HDL-cholesterol, |
| | | | GOT and GPT are |
| | | | selected) |
| | total cholesterol (TC) or | | 0.8034 (if TC and tb |
| | high-density lipoprotein | | are selected) |
| | (HDL) cholesterol | | 0.8186 (if |
| | | | HDL-cholesterol |
| | | | and tb are |
| | | | selected) |
| | liver function (GOT and | | 0.795 (if TC and alb |
| | GPT), total bilirubin (tb), | | are selected) |
| | and albumin (alb) | | 0.8164 (if |
| | | | HDL-cholesterol |
| | | | and alb are |
| | | | selected) |
| 8-1 | age, gender, race, BMI, | Asian | 0.8205 (if TC, GOT |
| | triglyceride and fasting | population | and GPT are |
| | plasma glucose (FPG) | | selected) |
| | | | 0.8198 (if |
| | | | HDL-cholesterol, |
| | | | GOT and GPT are |
| | | | selected) |
| | total cholesterol (TC) or | | 0.8076 (if TC and tb |
| | high-density lipoprotein | | are selected) |
| | (HDL) cholesterol | | 0.8075 (if |
| | | | HDL-cholesterol |
| | | | and tb are |
| | | | selected) |
| | liver function (GOT and | | 0.8037 (if TC and |
| | GPT), total bilirubin (tb), | | alb are selected) |
| | and albumin (alb) | | 0.8044 (if |
| | | | HDL-cholesterol |
| | | | and alb are |
| | | | selected) |
| 8-2 | age, gender, race, BMI, | Caucasian | 0.8228 (if TC, GOT |
| | triglyceride and fasting | population | and GPT are |
| | plasma glucose (FPG) | | selected) |
| | | | 0.8279 (if |
| | | | HDL-cholesterol, |
| | | | GOT and GPT are |
| | | | selected) |
| | total cholesterol (TC) or | | 0.8225 (if TC and tb |
| | high-density lipoprotein | | are selected) |
| | (HDL) cholesterol | | 0.8285 (if |
| | | | HDL-cholesterol |
| | | | and tb are |
| | | | selected) |
| | liver function (GOT and | | 0.8176 (if TC and |
| | GPT), total bilirubin (tb), | | alb are selected) |
| | and albumin (alb) | | 0.8254 (if |
| | | | HDL-cholesterol |
| | | | and alb are |
| | | | selected) |

### Relative Importance of Parameters in the XGBoost and Random Forests (RFs) Algorithms

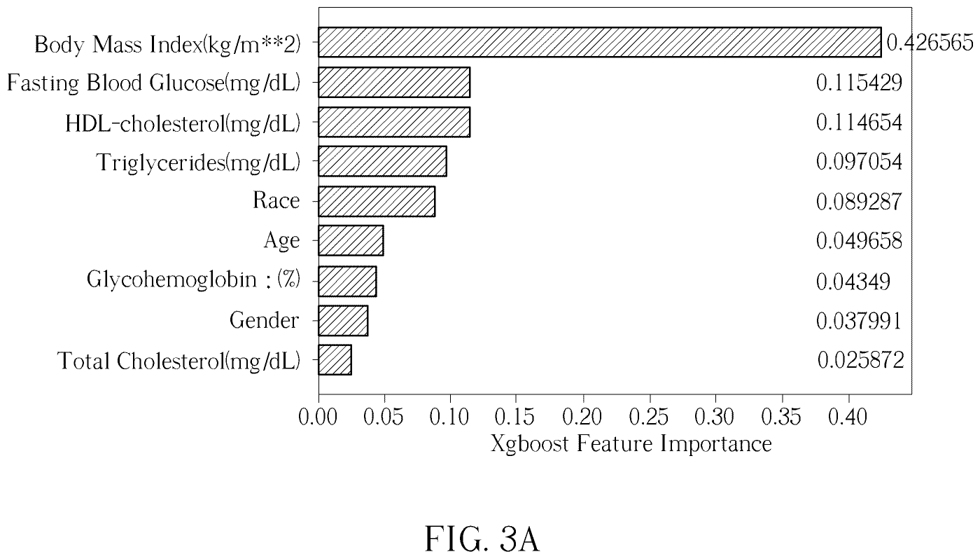

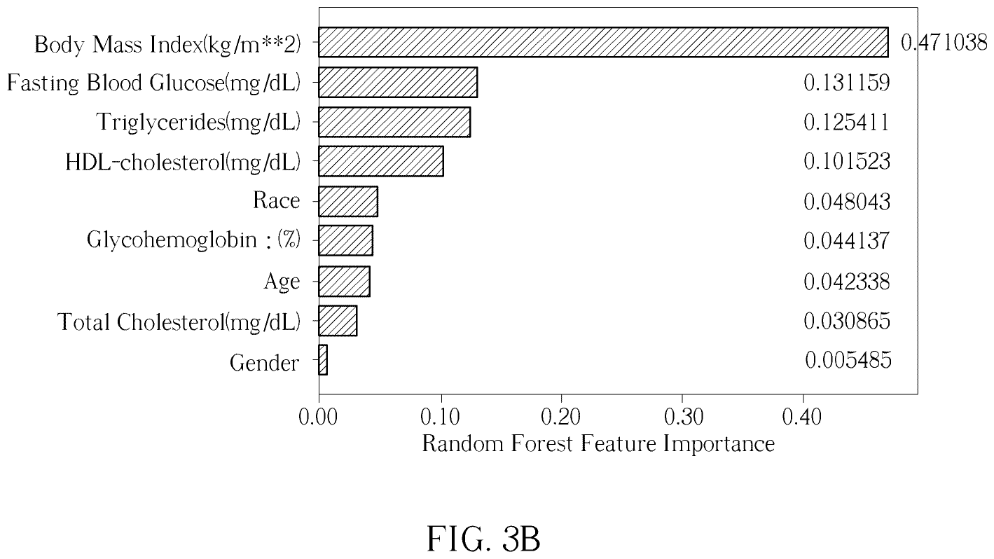

FIG. 3A and FIG. 3B show that the relative importance of each of the feature in the feature set of the present disclosure is studied in the XGboost and RFs prediction models, respectively. Among the features, the importance of all features is similar for the XGboost and RFs algorithms. BMI has the largest impact on IR (0.43 for XGboost and 0.47 for RFs algorithms) and its impact is significantly higher compared with the others. Aside from BMI, the variables that impact IR in descending levels of importance are as follows: FPG (0.12 for XGboost and 0.13 for RFs algorithms), HDL (0.11 for XGboost and 0.10 for RFs algorithms), and triglyceride (0.097 for XGboost and 0.13 for RFs algorithms).

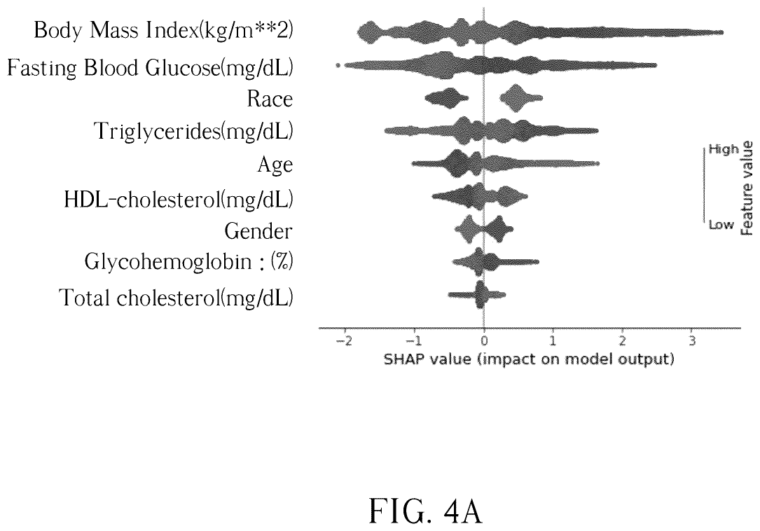

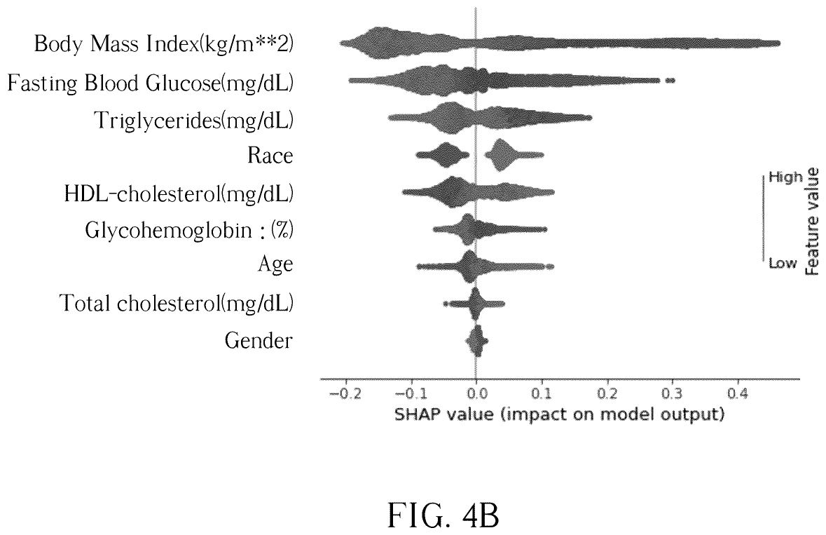

Referring to FIG. 4A and FIG. 4B, the features' relative contributions to the algorithm (i.e., SHAP value) in XGboost and the RFs algorithms showed either a positive or a negative association with IR (FIG. 4A for XGboost, and FIG. 4B for RFs). In both algorithms, all features were clearly differentiated into either positive or negative association with IR, and both algorithms gave similar patterns. Some features, such as BMI, FPG, triglyceride, gender, and HbAlc, show a positive impact on IR. Other features, such as race, age, HDL and total cholesterol, show a negative impact on IR.

Specifically, the SHAP (SHapley Additive exPlanations) is a method for attributing the contribution of each feature to the machine learning model's prediction, and calculating SHAP value involves averaging the contribution of each feature among all features. Hence, the SHAP value of each feature estimates the significance of each feature within the machine learning model in some embodiments of the present disclosure. As shown in FIG. 4A and FIG. 4B, the positive SHAP value is associated with an increased IR prediction, while the negative SHAP value is associated with a decreased IR prediction. The feature with a higher value is associated with red coloring, and the feature with a lower value is associated with blue coloring. There are 9 bands in FIGS. 4A and 9 bands in FIG. 4B, and the band of each feature represents the distribution of all the subjects of the present disclosure. Take the first band in FIG. 4A as an example, the higher the BMI shows the higher SHAP value, which indicates the higher the BMI, the higher the probability of IR prediction of the machine learning model. Further take the sixth band in FIG. 4A as an example, the lower HDL-cholesterol shows the higher SHAP value, which indicates the lower the HDL-cholesterol, the higher the probability of IR prediction of the machine learning model. In addition, the feature value of Asian race is 1, and the feature value of non-Asian race is 0.

### Impact of Features on IR Via Machine Learning Algorithms and Restricted Cubic Spline Analysis

An increasing trend is seen (the SHAP value increased with increasing features) with the following features: BMI, FPG, triglyceride, and HbAlc. A decreasing trend (SHAP values decreased with increasing features) is seen with the following features: HDL, age, and total cholesterol. All patterns of SHAP values from machine learning of the present disclosure are similar to the pattern of associations.

XGboost algorithm for predicting cardiovascular and all-cause mortality as validated externally by the fourth database of the present disclosure (i.e., Taiwan biobank)

Since the XGboost model has the best predictive value for IR, we applied the fourth data set of the present disclosure from the TWB database using XGboost algorithm. Here, participants from the TWB database are separated into predicted HOMA-IR (+) (IR) and HOMA-IR (−) (non-IR) groups.

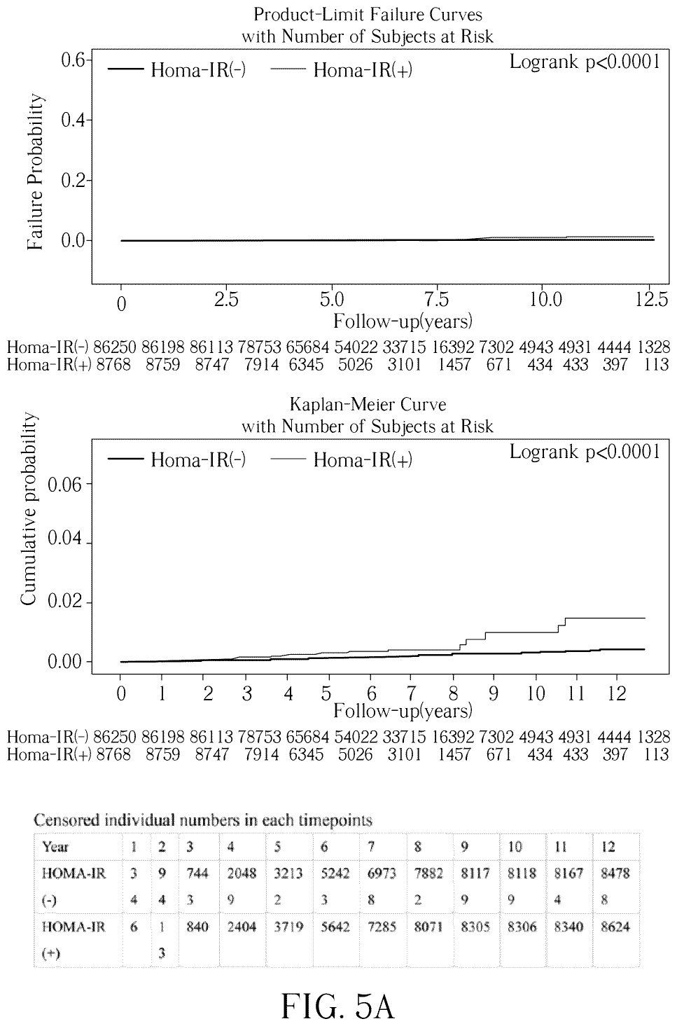

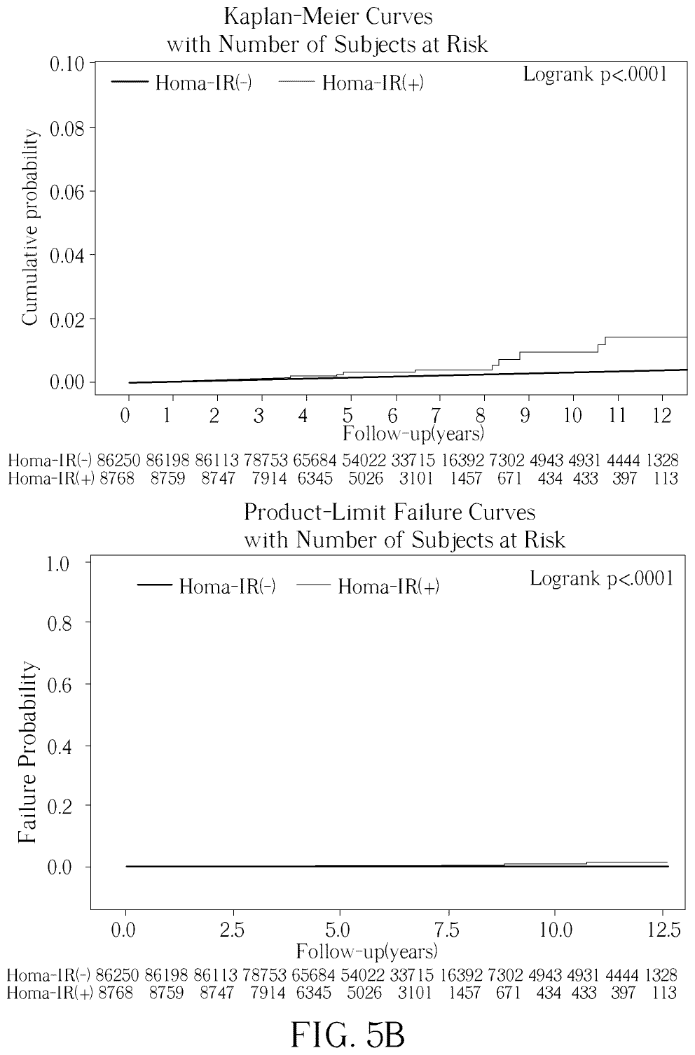

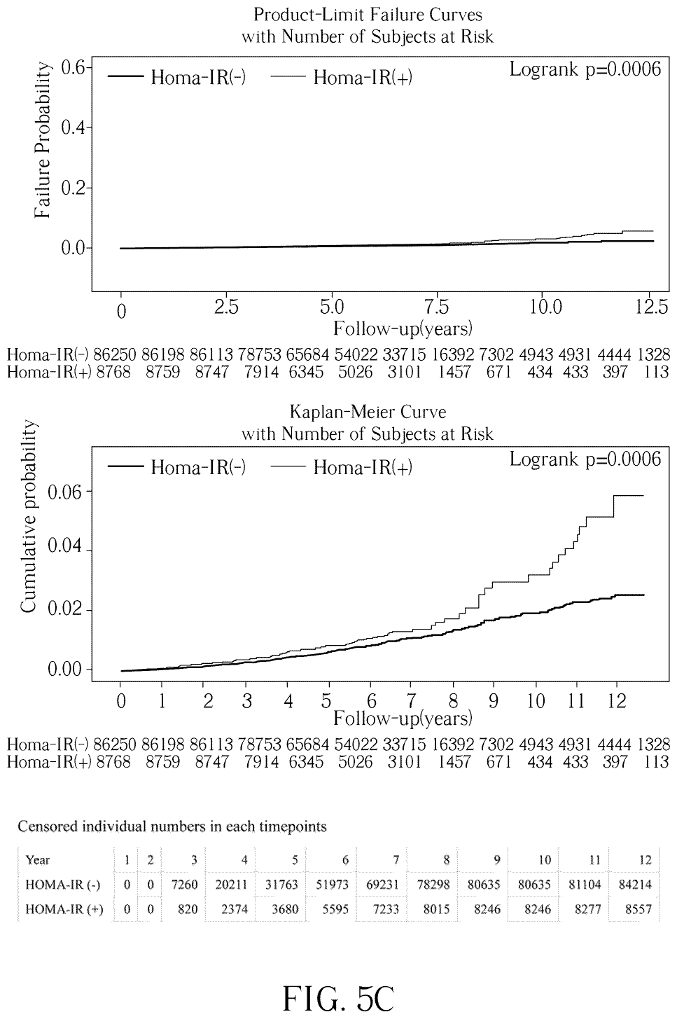

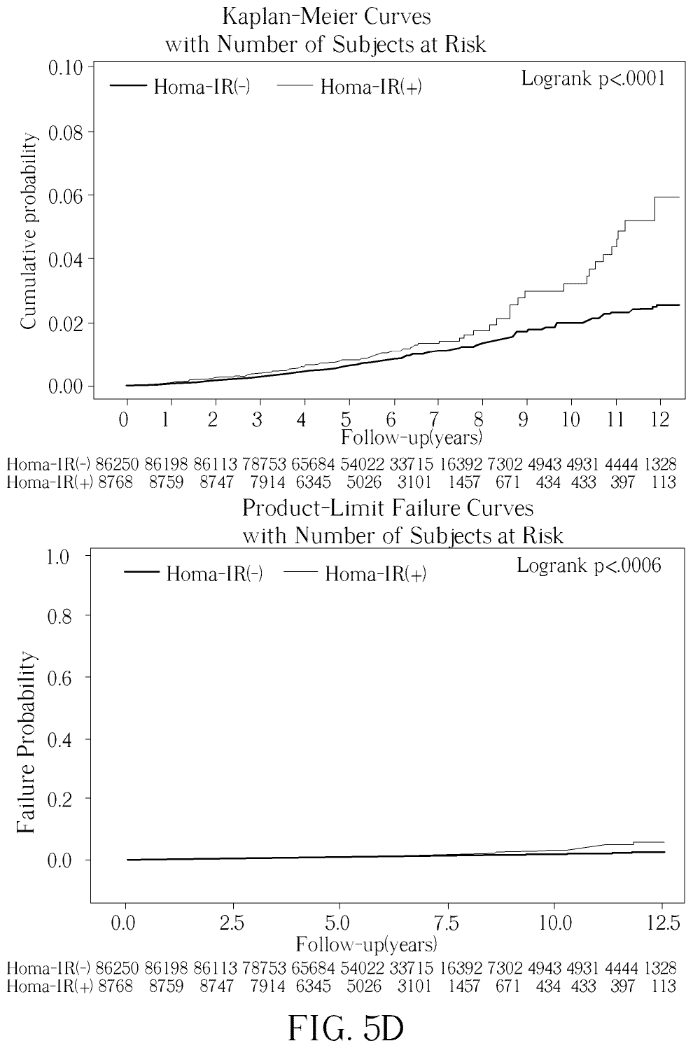

Referring to FIG. 5A, FIG. 5B, FIG. 5C, and FIG. 5D, the Kaplan-Meier survival curves (CV mortality in FIG. 5A and FIG. 5B; and all-cause mortality in FIG. 5C and FIG. 5D) show significant differences between the predicted IR and non-IR groups (p<0.0001 for CV mortality, and p=0.0006 for all-cause mortality). Therefore, the prediction of IR using the XGboost model has clear clinical implications specifically predicting worse CV and all-cause mortality. Possible associated factors by multivariate analysis are shown in Table 6.

TABLE 6
Univariate and multivariate analyses for factors associated
with CV and all-cause mortality in TWB database.

| Univariate analysis | | Multivariate analysis | |
|---|---|---|---|
| HR (95% CI) | p value | HR (95% CI) | p value |

Taiwan biobank database for predicting CV mortality

| | | | | | | |
|---|---|---|---|---|---|---|
| Age | 1.072 | (1.057-1.088) | <0.0001 | 1.069 | (1.054-1.085) | <0.0001 |
| Male v.s. Female | 3.657 | (2.771-4.825) | <0.0001 | 3.075 | (2.273-4.161) | <0.0001 |
| Body Mass | 1.098 | (1.065-1.132) | <0.0001 | 1.077 | (1.035-1.121) | 0.0002 |
| Index (kg/m²) | | | | | | |
| Glycohemoglobin: | 3.097 | (2.083-4.604) | <0.0001 | 1.573 | (1.009-2.453) | 0.0455 |
| (%) | .996-1.001) | 0.1666 | | | | |
| Fasting plasma | 1.043 | (1.027-1.059) | <0.0001 | 0.995 | (0.977-1.014) | 0.6078 |
| glucose (mg/dl) | | | | | | |
| HDL-cholesterol | 0.969 | (0.959-0.98) | <0.0001 | 0.99 | (0.976-1.004) | 0.1673 |
| (mg/dL) | | | | | | |
| Cholesterol | 0.999 | (0.995-1.003) | 0.5459 | 0.999 | (0.994-1.003) | 0.5037 |
| (mg/ dL) | | | | | | |
| Triglycerides | 1.001 | (1-1.002) | 0.0701 | 0.998 | (0.996-1.001) | 0.1666 |
| (mg/dL) | | | | | | |

Taiwan biobank database for predicting all-cause mortality

| | | | | | | |
|---|---|---|---|---|---|---|
| Age | 1.072 | (1.065-1.079) | <0.0001 | 1.074 | (1.067-1.081) | <0.0001 |
| Male v.s. | 2.196 | (1.95-2.474) | <0.0001 | 1.914 | (1.679-2.182) | <0.0001 |
| Female | | | | | | |
| Body Mass | 1.047 | (1.031-1.063) | <0.0001 | 1.029 | (1.01-1.049) | 0.0032 |
| Index (kg/m²) | | | | | | |
| Glycohemoglobin: | 1.708 | (1.43-2.041) | <0.0001 | 0.937 | (0.77-1.14) | 0.5147 |
| (%) | | | | | | |
| Fasting plasma | 1.029 | (1.022-1.037) | <0.0001 | 0.996 | (0.988-1.005) | 0.3826 |
| glucose (mg/dl) | | | | | | |
| HDL-cholesterol | 0.981 | (0.976-0.986) | <0.0001 | 0.996 | (0.99-1.002) | 0.196 |
| (mg/dL) | | | | | | |
| Cholesterol | 0.998 | (0.996-1) | 0.0142 | 0.996 | (0.994-0.998) | <0.0001 |
| (mg/dL) | | | | | | |
| Triglycerides | 1.001 | (1-1.001) | <0.0001 | 1 | (1-1.001) | 0.2482 |
| (mg/dL) | | | | | | |

In Table 7, in both univariate analysis and multivariate analysis, comparing HOMA-IR>2.5-≤12.5, there are significantly higher hazard ratio (HR) for all-cause and CV mortality.

TABLE 7
Proportional HR model for time-to-event analysis.

| | |
|---|---|
| HR (95% CI) | p value |

Univariate analysis
All-cause mortality

| | | | |
|---|---|---|---|
| HOMA-IR >2.5 vs. ≤2.5 | 1.443 | (1.168-1.783) | 0.0007 |

CV mortality

| | | | |
|---|---|---|---|
| HOMA-IR >2.5 vs. ≤2.5 | 2.394 | (1.620-3.538) | <0.0001 |

Multivariate analysis
All-cause mortality

| | | | |
|---|---|---|---|
| Age | 1.069 | (1.061-1.077) | <0.0001 |
| Female gender | 0.454 | (0.395-0.522) | <0.0001 |
| HOMA-IR >2.5 vs. ≤2.5 | 1.474 | (1.192-1.822) | 0.0003 |

CV mortality

| | | | |
|---|---|---|---|
| Age | 1.077 | (1.059-1.095) | <0.0001 |
| Female gender | 0.302 | (0.219-0.415) | <0.0001 |
| HOMA-IR >2.5 vs. ≤2.5 | 2.4 | (1.620-3.556) | <0.0001 |

Compared to the Framingham score for CV mortality (shown in Table 8), the XGboost had less predictive power (0.547 vs. 0.643 and 0.557 of concordances). However, in Table 9, for condition 1 (cutoff value: 10% Framingham score estimation of 10-year CV risk), in the group of HOMAIR>2.5 (XGboost)+& ≤10% (Framingham Score), there is still increased risk for mortalities (all-cause mortality: 1.556 (95% confidence interval (CI): 1.186-20.41), p=0.0014) (CV mortality: 3.374 (95% CI: 2.051-5.552, p<0.0001)) even though the risk prediction of Framingham Score is ≤10%. Similarly, in condition 2 (cutoff value: 20% Framingham score estimation of 10-year CV risk), in the group of HOMAIR>2.5 (XGboost)+& ≤20% (Framingham Score), there is still increased risk for mortalities (all-cause mortality: 1.433 (95% CI: 1.141-1.8, p=0.002)) (CV mortality: 2.655 (95% CI: 1.752-4.023, p<0.0001)) even though the risk prediction of Framingham Score is ≤20%.

TABLE 8
C index to compare the predictive algorithm (XGboost)
and Framingham Score for CV mortality.

| Predictive power by two | CV mortality predictive power |
|---|---|
| methods | (C index) |
| IR+ predicted by XGboost | Concordance = 0.547 (se = 0.024) |
| Framingham Score for moderate | Concordance = 0.643 (se = 0.021) |
| risk (>10%/10 yr) | |
| Framingham Score for moderate | Concordance = 0.557 (se = 0.015) |
| risk (>20%/10 yr) | |

TABLE 9
Misclassification statistics for XGboost and Framingham Score for CV mortality.

| | |
|---|---|
| HR (95% CI) | p value |

Condition 1. Cutoff value: 10% Framingham score estimation of 10-year CV risk

| | | | |
|---|---|---|---|
| All-cause mortality | | | |
| Age | 1.065 | (1.057-1.074) | <0.0001 |
| Female gender | 0.491 | (0.422-0.571) | <0.0001 |
| HOMAIR ≤2.5 (XGboost) & ≤10% | 1 | (reference) | |
| (Framingham Score) | | | |
| HOMAIR >2.5 (XGboost) & ≤10% | 1.556 | (1.186-20.41) | 0.0014 |
| (Framingham Score) | | | |
| HOMAIR ≤2.5 (XGboost) & >10% | 1.3 | (1.074-1.573) | 0.0071 |
| (Framingham Score) | | | |
| HOMAIR >2.5 (XGboost) & >10% | 1.609 | (1.609-1.152) | 0.0052 |
| (Framingham Score) | | | |
| CV mortality | | | |
| Age | 1.604 | (1.045-1.084) | <0.0001 |
| Female gender | 0.383 | (0.27-0.545) | <0.0001 |
| HOMAIR ≤2.5 (XGboost) & ≤10% | 1 | (reference) | |
| (Framingham Score) | | | |
| HOMAIR >2.5 (XGboost ) & ≤10% | 3.374 | (2.051-5.552) | <0.0001 |
| (Framingham Score) | | | |
| HOMAIR ≤2.5 (XGboost) & >10% | 2.223 | (1.48-3.339) | 0.0001 |
| (Framingham Score) | | | |
| HOMAIR >2.5 (XGboost) & >10% | 2.70 | (1.431-5.286) | 0.0024 |
| (Framingham Score) | | | |

Condition 2. Cutoff value: 20% Framingham score estimation of 10-year CV risk

| | | | |
|---|---|---|---|
| All-cause mortality | | | |
| Age | 1.067 | (1.059-1.075) | <0.0001 |
| Female gender | 0.471 | (0.408-0.544) | <0.0001 |
| HOMAIR ≤2.5 (XGboost) & ≤20% | 1 | (reference) | |
| (Framingham Score) | | | |
| HOMAIR >2.5 (XGboost) & ≤20% | 1.433 | (1.141-1.8) | 0.002 |
| (Framingham Score) | | | |
| HOMAIR ≤2.5 (XGboost) & >20% | 1.408 | (1.025-1.933) | 0.0346 |
| (Framingham Score) | | | |
| HOMAIR >2.5 (XGboost) & >20% | 2.102 | (1.229-3.595) | 0.0067 |
| (Framingham Score) | | | |
| CV mortality | | | |
| Age | 1.071 | (1.053-1.09) | <0.0001 |
| Female gender | 0.331 | (0.237-0.462) | <0.0001 |
| HOMAIR ≤2.5 (XGboost) & ≤20% | 1 | (reference) | |
| (Framingham Score) | | | |
| HOMAIR >2.5 (XGboost) & ≤20% | 2.655 | (1.752-4.023) | <0.0001 |
| (Framingham Score) | | | |
| HOMAIR ≤2.5 (XGboost) & >20% | 2.45 | (1.418-4.232) | 0.0013 |
| (Framingham Score) | | | |
| HOMAIR >2.5 (XGboost) & >20% | 2.095 | (0.657-6.682) | 0.2115 |
| (Framingham Score) | | | |

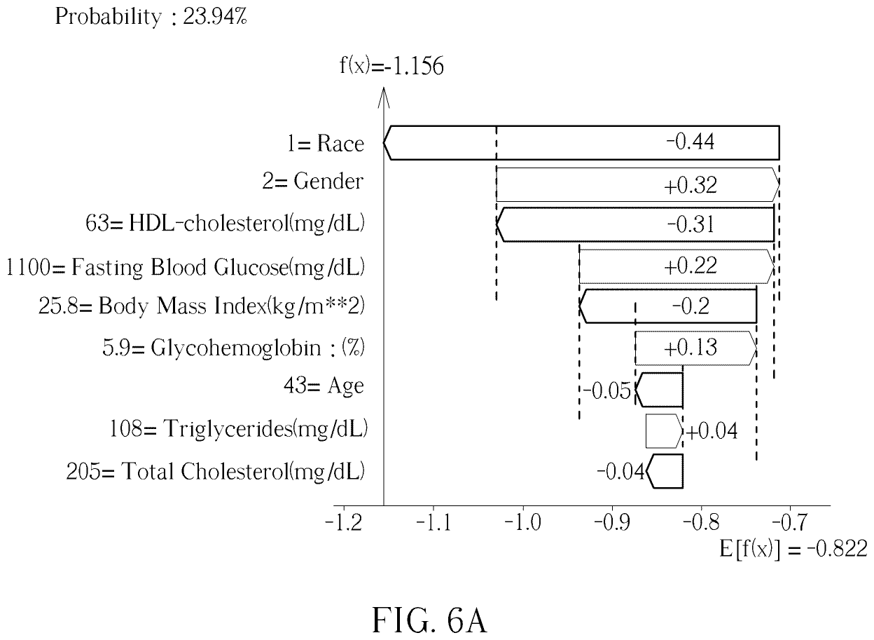

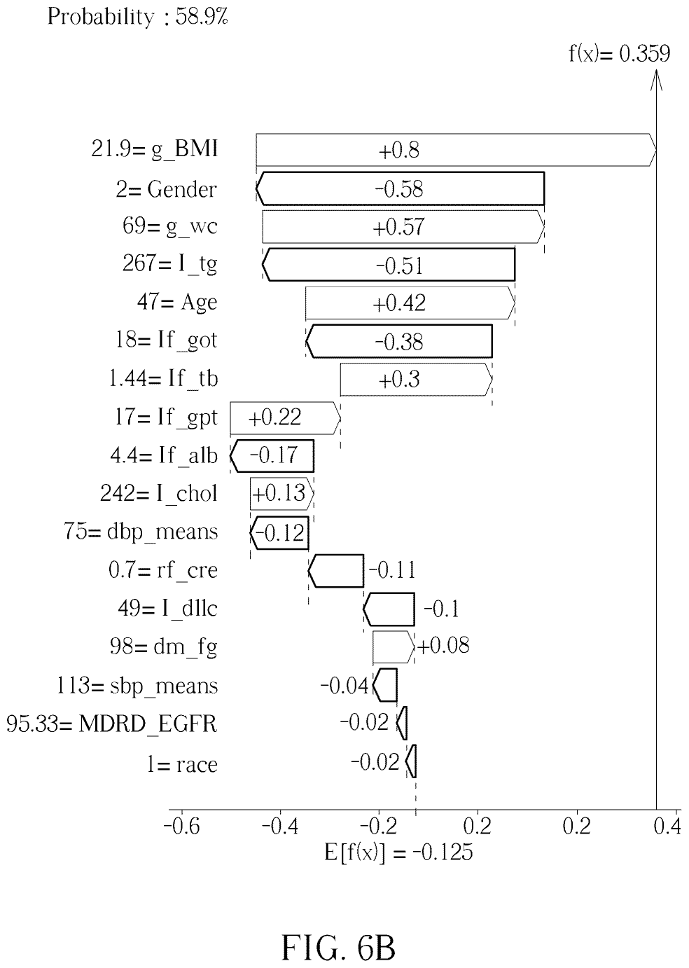

Referring to FIG. 6A and FIG. 6B, the result of the personalized IR risk (FIG. 6A), personalized β-cell deficiency risk (FIG. 6B), and explanation of a feature set are illustrated. Specifically, wc is an abbreviation of waist circumference; tg is an abbreviation of triglyceride; got is an abbreviation of glutamate oxaloactate transaminase; tb is an abbreviation of total bilirubin; gpt is an abbreviation of glutamate pyruvate transaminase; alb is an abbreviation of albumin; chol is an abbreviation of total cholesterol; dpb is an abbreviation of diastolic blood pressure; cre is an abbreviation of creatinine; hdlc is an abbreviation of high-density lipoprotein cholesterol; fg is an abbreviation of fasting blood glucose; sbp is an abbreviation of systolic blood pressure; MDRD is an abbreviation of modification of diet in renal disease equation; and EGFR is an abbreviation of estimated glomerular filtration rate. The patients and the medical personnel may be able to adjust the medical treatment or personal lifestyle according to the aforementioned result.

In some embodiments, a model building and optimization module of the present disclosure builds a machine learning model of the present disclosure based on the feature set of the third data set of the present disclosure to predict the pancreatic β-cell function of the non-diabetes mellitus patient, wherein the feature set comprises age, gender, race, body mass index (BMI), fasting blood/plasma glucose (fg/FBG/FPG), triglyceride (tg), total cholesterol (TC/chol) high-density lipoprotein (HDL) cholesterol (HDL-cholesterol/hdlc), glutamate oxaloactate transaminase (got), glutamate pyruvate transaminase (gpt), waist circumference (wc), total bilirubin (tb), albumin (alb), systolic blood pressure (sbp), diastolic blood pressure (dbp), estimated glomerular filtration rate (eGFR), and/or creatinine (cre).

In some embodiments, the machine learning of the present disclosure using the third data set from the third database (i.e., a combined database of the NHANES and MJ databases) is applied to find a good model for predicting IR and β-cell function in a non-diabetic population with high diagnostic performance (0.88 of AUC) based on a feature set of the present disclosure that is easily obtained from patients.

In some embodiments, predicted IR via this trans-ethnic algorithm was associated with clinical implications validated using the TWB database, specifically: worse CV and all-cause mortality. In addition, even though lower predictive power (lower C index) for risk of CV mortality compared to Framingham score, patients with

HOMA-IR>2.5 (under lower risk prediction for CV by Framingham score estimation), are still risky for higher CV and all-cause mortality. The Framingham score cannot fully predict CV and all-cause mortality especially in the population without DM of the present disclosure. This also echoes the importance of the present disclosure.

A number of machine learning studies related to DM have been published. However, based on the findings of the UKPDS, ADVANCE, VADT, and ACCORD studies, sugar control should be implemented early to obtain good vascular outcomes. Based on the DM spectrum, it can be inferred that identifying IR and β-cell function is crucial. However, a patient's insulin level, which is not collected routinely in clinical practice, is needed to calculate the HOMA-IR and HOMA-R. Therefore, devising a predictive model for IR and β-cell function (without empirical insulin value) is important.

The predictive machine learning model of the present disclosure has two outstanding strengths: it has better predictive power (highest AUC), and is more generalizable (only easily obtained features, and trans-racial application).

The major impact of fasting plasma insulin level on HOMA-IR also indicates the importance of the present disclosure. First, there is no data on fasting plasma insulin levels to calculate HOMR-IR in the present disclosure, even though it has the largest impact on IR. Second, in different racial populations (such as Asian and Caucasian), the values of HOMA-IR are significantly different (NHANES population (Caucasian): 2.84 (2.77-2.91), MJ database (Asian): 1.92±1.26). This difference of IR is mostly due to fasting plasma insulin level (12.1±10.35 mIU/L in NHANES vs. 7.67±4.76 in MJ database), rather than FPG level (91.54 mg/dL in NHANES vs. 100.07±8.3 in MJ database). The present disclosure aimed to train a machine learning model that can be used generally for different races to predict IR without the value of fasting plasma insulin.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A system for predicting insulin resistance and/or pancreatic β-cell function of a subject in need thereof, comprising:
a database configured to provide a data set;
a feature extraction module configured to collect and process features from the data set to generate a feature set regarding the subject, wherein the feature set comprises age, gender, race, and body mass index of the subject; and
a model building and optimization module configured to build a machine learning model based on the feature set to predict the insulin resistance and/or the pancreatic β-cell function of the subject.

2. The system of claim 1, wherein the feature set further comprises fasting blood glucose, and/or glycohemoglobin of the subject.

3. The system of claim 2, wherein the feature set further comprises total cholesterol and/or high-density lipoprotein cholesterol of the subject.

4. The system of claim 1, wherein the feature set further comprises triglyceride of the subject.

5. The system of claim 4, wherein the feature set further comprises total cholesterol and/or high-density lipoprotein cholesterol of the subject.

6. The system of claim 5, wherein the feature set further comprises fasting blood glucose and at least one selected from the group consisting of glutamic oxaloacetic transaminase, glutamic pyruvic transaminase, total bilirubin, and albumin of the subject.

7. The system of claim 1, wherein the feature set further comprises glutamic oxaloacetic transaminase, glutamic pyruvic transaminase, total bilirubin, and/or albumin of the subject.

8. The system of claim 7, wherein the feature set further comprises fasting blood glucose and at least one selected from the group consisting of total cholesterol and high-density lipoprotein cholesterol of the subject.

9. The system of claim 1, wherein the machine learning model is trained by the features labeled with outcome related to the insulin resistance and/or a decline of β-cell function of the subject.

10. The system of claim 1, wherein the database comprises:
a first database derived from a first population of the subject; and
a second database derived from a second population of the subject,
wherein the race of the first population is different from the race of the second population.

11. A method for predicting insulin resistance and/or pancreatic β-cell function of a subject in need thereof, comprising:
configuring a database to provide a data set;
configuring a feature extraction module to collect and process features from the data set to generate a feature set regarding the subject, wherein the feature set comprises age, gender, race, and body mass index of the subject; and
configuring a model building and optimization module to build a machine learning model based on the feature set to predict the insulin resistance and/or the pancreatic β-cell function of the subject.

12. The method of claim 11, wherein the feature set further comprises fasting blood glucose, and/or glycohemoglobin of the subject.

13. The method of claim 12, wherein the feature set further comprises total cholesterol and/or high-density lipoprotein cholesterol of the subject.

14. The method of claim 11, wherein the feature set further comprises triglyceride of the subject.

15. The method of claim 14, wherein the feature set further comprises total cholesterol and/or high-density lipoprotein cholesterol of the subject.

16. The method of claim 15, wherein the feature set further comprises fasting blood glucose and at least one selected from the group consisting of glutamic oxaloacetic transaminase, glutamic pyruvic transaminase, total bilirubin, and albumin of the subject.

17. The method of claim 11, wherein the feature set further comprises glutamic oxaloacetic transaminase, glutamic pyruvic transaminase, total bilirubin, and/or albumin of the subject.

18. The method of claim 17, wherein the feature set further comprises fasting blood glucose and at least one selected from the group consisting of total cholesterol and high-density lipoprotein cholesterol of the subject.

19. The method of claim 11, wherein the model building and optimization module builds the machine learning model based on the feature set to predict the insulin resistance and/or the pancreatic β-cell function of the subject by classifying the subject into an insulin resistance group, a non-insulin resistance group, β-cell deficiency group, and a non-β-cell deficiency group, and generating a corresponding predictive value and a classification performance value thereof, wherein the classification performance value is an area under curve of a receiver operating characteristic curve of the machine learning model.

20. The method of claim 11, wherein the machine learning model is trained by the features labeled with outcome related to the insulin resistance and/or a decline of β-cell function of the subject.

21. The method of claim 11, wherein the database comprises:
a first database derived from a first population of the subject; and
a second database derived from a second population of the subject,
wherein the race of the first population is different from the race of the second population.

22. A computer readable medium storing a computer executable code, upon executed, the computer executable code implement the method according to claim 11.
